(19) United States
(12) Patent Application Publication
(10) Pub. No.: US 2011/0040086 A1
(43) Pub. Date: Feb. 17, 2011

(54) Novel Beta-Lactam Antibiotics, Methods for Their Production, and Their Use

(75) Inventors: Wolf-Dieter Juelich, Greifswald (DE); Ulrike Lindequist, Greifswald (DE); Annett Mikolasch, Greifswald (DE); Sabine Witt, Greifswald (DE); Frieder Schauer, Lubmin (DE); Roland Ohme, Berlin (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: DRITTE PATENTPORTFOLIO BETEILIGUNGSGESELLSCHAFT MB, Schoenefeld/Waltersdorf (DE)

(21) Appl. No.: 12/525,322

(22) PCT Filed: Jan. 31, 2008
(86) PCT No.: PCT/EP2008/051214

§ 371 Date: Nov. 23, 2009

(30) Foreign Application Priority Data
Jan. 31, 2007 (EP) 07101519.2

(51) Int. Cl. C07D 501/00; C07D 471/02; C12P 35/00; C12P 17/12; C07C 279/02

(52) U.S. Cl. 540/215; 540/205; 435/47; 435/122; 564/233

(57) ABSTRACT

The invention relates to novel antimicrobial agents that are based on β-lactam derivatives and are produced by reacting previously known β-lactam derivatives with polyphenol oxidase substrates under the influence of free radicals and by forming salts of any β-lactam derivatives with polyhexamethylene biguanide hydrogen carbonate. Said novel compounds are suitable as an antibiotic.

The invention relates to novel antimicrobial agents based on β-lactam derivatives and their use as antibiotics.

### PRIOR ART

β-lactam antibiotics, especially the cephalosporins, belong to the most used antibiotics. Like the structurally closely related carbacephems and penicillins, cephalosporins inhibit the synthesis of the bacterial cell wall and have a bactericide effect only in the growth phase of the bacteria. The antibiotic group of cephalosporins has been intensively cultivated. Clinically used derivatives are usually derived from the base compound 7-amino cephalosporanic acids, wherein changes were performed on the base compound as an R1 substitution in position 7, as an R2 substitution in position 3, and also, for cephamycins, by an additional methoxy group in position 7.

Cephalosporins and carbacephems offer better possibilities for structural modification and effectiveness optimization than penicillins, as evidenced by the large number of synthesized cephalosporin derivatives (Gräfe U., Biochemie der Antibiotika [Biochemistry of Antibiotics], Spektrum Akademischer Verlag, Heidelberg, Berlin, N.Y., 1992).

In each group of the β-lactam antibiotics there are proven substances that are to be preferred for certain indications, due to their effectiveness spectrum and their pharmacokinetic properties.

No single β-lactam derivative could previously be considered as universally applicable. Therefore, a multi-purpose molecular design is essential, in order to arrive at the derivatives suitable for the intended application.

However, resistances have also emerged relative to these initially effective antibiotics, especially for staphylococci (methicillin resistant S.-aureus strains=MRSA). The percentage of resistant strains in clinical isolates is constantly growing in all highly industrialized countries; in the USA, Japan, and China, it currently equals greater than 70%. Hospital infections with multi-resistant staphylococci are increasingly difficult to overcome for patients with a weakened immune response. Therefore, the development of antibiotics with effectiveness against multi-resistant staphylococci is of great importance.

Esterification of the carboxyl group of cephalosporins corresponds to the prior art, e.g.: M. Murakami, M. Hajima, F. Takami, M. Yoshioka (Heterocylces, 31:2055-264, 1990). The esterification leads, among other things, to derivatives that can be better reabsorbed. Here, enzymes could be used, in order to allow a reaction under conservative conditions. Lipases catalyze the esterification with a wide pallet of substrates (Ching et al., Angew. Chem. 101:711-724, 1989).

Poly-hexamethylene-biguanide hydrochloride of Formula 1

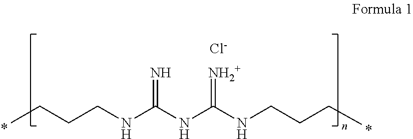

is known as a microbiocide and has been introduced under the names Vantocil IR, Polihexanid, Lavasept R, or PHMB-HCl in practice for desinfection and antiseptics and is used especially as a wound antiseptic, furthermore as an adjuvant for treating wounds for the surgical care of acute and chronic bone and soft-tissue infections. It involves a polymer mixture of various molecular weight ranges, whose separation could be detected up until now only using chromatography, but could not be performed preparatively. The known microbiocide effects therefore always relate to the polymer mixture and are not optimized. In medicine, until now, mixtures made from polymers with a different number of sub-units as hydrochloride have been used exclusively. Polymers with 4 to 7 units and a molecular weight of 900 to 1300 g/mol are typical. The generation of pure polymer fractions has not been successful up until now. The purification of the polymer mixture for purposes of medical application is difficult and expensive.

A combination of β-lactam antibiotics and polyhexamethylene biguanides has not been known up until now.

In summary, it can thus be stated that the most important disadvantage of β-lactam antibiotics is their increasing limitation in effectiveness due to the buildup of resistance in the bacteria to be combated. In veterinary medicine, infections in farm animals with multi-resistant staphylococci are becoming more and more common.

Due to the increasing problem of resistance, there is a great need for new antibiotic agents that is not met by known active ingredients.

### Problem of the Invention

The problem of the present invention is therefore to make available novel active ingredients. The invention is based especially on the problem of meeting the need arising due to the increasing development of resistance of bacteria with respect to conventional antibiotics in human and veterinary medicine through antibiotics that are altered in structure and effectiveness and that are derived from clinically proven active ingredients.

### Solution of the Problem

The problem is solved according to the features of the claims. According to the invention, new, previously unknown active ingredients from the group of β-lactam antibiotics are provided. In addition, novel compounds are produced through the formation of salts of β-lactam antibiotics with cations, which themselves have an active ingredient character, as well as through refinement of these active ingredients by subsequent chemical reactions.

In the Structural Formulas 4, 7, and 8, the novel active ingredients according to the invention are illustrated.

The subject matter of the invention also includes a method for producing the β-lactam antibiotics according to the invention, including intermediate products. For solving the problem, two different paths were proposed that could be advantageously combined according to the invention.

### Path 1:

Surprisingly, β-lactam antibiotics according to Structural Formula 3, which are made from a derivative of 6-aminopenicillanic acids or 7-aminocephalosporanic acids as anionic component X⁻ and derivatives of the polyhexamethylene biguanide as cationic components, have proven to be highly effective. The active ingredient according to the invention causes inhibition in all tested, multireistant bacteria strains, even in strains in which both the cationic active ingredient as the hydrochloride and also the anion (i.e., antibiotic without cationic component) are ineffective.

In microbiological studies, it could be shown that even MRSA germs, which are very difficult to combat and often present problems in clinics, are inactivated by the novel active ingredients. The active ingredients according to the invention according to Formula 3 are therefore suitable for the anti-infectious treatment of acute and chronic wounds, including application for irrigation-suction drainage and for the anti-infectious lavage of visceral cavities.

In these previously unknown compounds according to Structural Formula 3 or 7, the excellent microbiocidal properties of the β-lactam antibiotics are combined with additional effects that are highly interesting for the practice. Formulas 4 to 9, as well as the Reaction Equations 10 and 11 with the formulas, are found before the embodiments. Through the combination with the surface-active cation, exciters are reached at positions that are especially difficult to access. In the compounds according to the invention according to Formula 7, hydrophilic guanide and β-lactam groups stand opposite lipophilic hydrocarbon chains and explain the tenside properties of the novel active ingredients. Due to these tenside properties, the compounds according to the invention could also be effective in biofilms.

Compounds of this type could be obtained from hydrogen carbonates of the polyhexamethylene biguanide according to Structural Formula 2 that has not been described up until now. In this way, advantage is taken of the fact that the hydrogen carbonate is soluble in water with much more difficulty than commercially available hydrochloride. In this way, initially polyhexamethylene biguanide hydrochloride is converted in aqueous solution with alkali hydrogen carbonate to polyhexamethylene biguanide hydrogen carbonate according to Formula 2

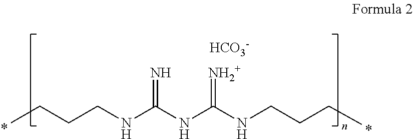

and from this with an acid, a polyhexamethylene biguanide derivative according to Formula 3 is formed:

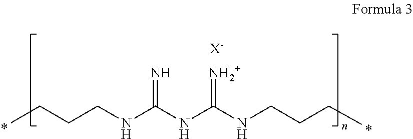

If this precipitation is performed gradually by the partial addition of carbonate or hydrogen carbonate, then the higher molecular weight fractions precipitate out first. This principle leads, on one hand, to a method for separating biguanides into molecular weight ranges for which protection is also sought with this patent. The separation of the polymer mixture into fractions of different molecular weight ranges is advantageous for many fields of application.

In particular, this principle can be used advantageously in the production of novel derivatives of β-lactam antibiotics, not previously described. Through the fractionated precipitation with sodium hydrogen carbonate, on one hand, and the substitution of the β-lactams, on the other hand, the resulting product could be adapted to the requirements of various applications, in particular the distribution behavior could be varied systematically. By the substitution of the β-lactams, the distribution behavior, and thus the logP value, changes. The introduction of a parachinoid substitution (logP values <or >0) into antibiotics (logP values <0) corresponding to the embodiments is associated with an increase of the distribution coefficient (logP value >0).

An advantageous application of the insoluble hydrogen carbonate according to Structural Formula 2 and/or the salts obtained from this substance according to Structural Formula 3 with antimicrobial effective fatty acid derivatives and/or β-lactam antibiotics as an anion, if desired, is given for the antimicrobial material of the absorbent core of wound dressings. The particular advantage given for a material of the absorbent core with a substance according to Structural Formula 2 in a concentration of 0.01 to 0.03%, especially advantageously 0.02%, is that the germicidal effect on the absorbent dressing is limited. Therefore, despite antimicrobial material, it is not restricted to categorization as a medical product.

A complete suppression of germ growth in and below the wound dressing is achieved based on the insolubility of the substance according to Structural Formula 2, not previously described, but diffusion into the wound is stopped. However, a zone of inhibition of only 0.93±0.73 mm (n=7) was observed, i.e., diffusion in regions around the wound dressing was minimal. Greater diffusion into the wound begins only at concentrations >0.04%.

In this way, a problem is solved, which arises in uncoated, absorbent wound dressings and in wound compresses with an absorbent core. In wound dressings and especially in the absorbent core, a strong germ propagation is produced that is, on one hand, dangerous to the healing process and that could lead, on the other hand, to germ carryover.

By reaction of the polyhexamethylene biguanide hydrogen carbonate with derivatives of the 6-aminopenicillanic acids or the 7-aminocephalosporanic acids, according to the invention, many previously unknown salts of the hexamethylene biguanide are accessible in a simple way preparatively with antimicrobially highly effective anions. In this way, antimicrobial compounds are obtained, which could have many uses.

### Path 2:

On the other hand, novel β-lactam antibiotics according to the general Formula 4 or 8 (claim 1) are obtained when substrates of polyphenol oxidases, in a particularly advantageous way, substrates according to Formula 6 or Formula 9, are linked under the influence of free radicals with β-lactam antibiotics, in a particularly advantageous way, with derivatives according to Formula 5. The hydroxy groups of the substrates of the polyphenol oxidases could be arranged according to Formula 6 and Formula 9 in the para or ortho position.

The amination of 2,5-dihydroxybenzoic acid derivatives with Laccase EC1-10.3.2 (classification according to International Enzyme Nomenclature; Enzyme Nomenclature, Academic Press, Inc., 1192, pp. 24-154), which leads to broad derivation possibilities, is especially preferred for the synthesis of the novel active ingredients.

The amination with catechols is limited, according to the invention, to active ingredients according to Formula 8.

The active ingredients according to the invention are characterized in that the novel substitutes change the application properties, without influencing functional groups. The active ingredients according to the invention therefore exhibit advantages to previously known β-lactam antibiotics, namely a high bactericide effect with low toxicity.

The radicals needed for the synthesis of the novel active ingredients according to the invention can be generated in biological, chemical, and/or physical ways. Especially preferred are radicals that are generated through the use of supernatants of ligninolytic fungi and/or from the supernatants of isolated, radical-forming enzymes. Preferably, radical-forming enzymes of the classification E C 1.10.3.2 and peroxidases of the classification EC 1.11.17, monophenol monooxygenase EC 114.99.1, and/or ascorbat oxidase EC 1.10.3.3 are used. As an example, Laccase of Trametes sp. can be used for the synthesis of the novel active ingredients.

Starting from the novel active ingredients according to Formulas 4 and 8, additional novel antibiotics can be obtained by esterification of the carboxyl group known in the prior art. In this way, enzymes can be used (e.g., lipases), in order to allow a reaction under conservative conditions (low temperatures, normal pressure).

The protective effect of the novel active ingredients according to Structural Formulas 4 and 8 in S. aureus Sepsis mouse model is especially surprising. In a survival test after i.p. infection of mice, without effective treatment, 100% of the animals die. The two-time application of active ingredients according to the invention after 30 min and 6 h, however, lead to complete recovery of the animals without permanent, visible damage. An identical therapy success is achieved when the antibiotic according to the invention is administered after 6 and 20 h. The novel active ingredients according to the invention expand the therapy possibilities in the treatment of bacterial infections, because effectiveness against germs that have become multi-resistant can also be proven.

The active ingredients of the general Formulas 3, 4, and 8 can be used both by themselves and also in combination with other active ingredients.

Particular advantages are achieved when the novel active ingredients of the general Formula 7 are used, because, in this case, compounds that are very effective against multi-resistant germs are obtained.

In addition, the invention makes available a method for the purification of polyhexamethylene biguanides, characterized in that polyhexamethylene biguanide hydrochloride (Structural Formula 1) is precipitated in aqueous solution with alkali hydrogen carbonate, the precipitate is separated from the mother liquor, and is converted back with hydrochloric acid into purified product according to Structural Formula 1.

For the production of additional novel salts of the polyhexamethylene biguanide, practically all inorganic and organic acids are suitable whose acidic strength exceeds that of the hydrogen carbonate. In this way, additional applications are opened up.

The application of the active ingredients according to Structural Formula 3 with antimicrobially effective fatty acid derivatives and/or β-lactam antibiotics as anion leads to special advantages for local application. An application on the udder as a mastitis prophylaxis in cattle is especially advantageous. Through use in preparations for applications on the udder, a mastitis can be treated and/or the transmission of staphylococcus infections can be prevented and thus contamination of the milk can be avoided.

Additional preparations suitable for local applications are obtained when the novel β-lactam antibiotics are mixed with lipids and are transformed by high-pressure cracking homogenization into micro-particles and nano-particles.

In summary, the invention shall be briefly described again:

β-lactam antibiotics were prepared for the first time according to Structural Formula 3, which can be obtained through the formation of salts from derivatives of the 6-aminopenicillanic acids or the 7-aminocephalosporanic acids as the anionic component X and derivatives of the polyhexamethylene biguanide as the cation. In addition, β-lactam antibiotics were produced according to Structural Formula 4, which can be obtained from commercially available active ingredients according to Structural Formula 5 by reaction with active ingredients according to Structural Formula 6. Furthermore, β-lactam antibiotics according to Structural Formula 8, which can be obtained from commercially available active ingredients according to Structural Formula 5 by reaction with active ingredients according to Structural Formula 9 are the subject matter of the present invention.

A method for the separation of polyhexamethylene biguanide into molecular weight ranges will be described, characterized in that polyhexamethylene biguanide hydrochloride is precipitated out in aqueous solution with alkali hydrogen carbonate, wherein the precipitation takes place partially and step by step, and fractionation into narrow molecular weight ranges of the polymers is thereby performed. The active ingredients according to Structural Formula 3 are characterized in that polyhexamethylene biguanide hydrochloride is precipitated in a fractionated manner in aqueous solution with alkali hydrogen carbonate to polyhexamethylene biguanide hydrogen carbonate, and the precipitation products are converted with antibiotics that exceed the hydrogen carbonate in their acidic strength. It is also possible that the anionic component is an antimicrobially effective fatty acid. The solubility and distribution behavior of the active ingredients can be varied through fractionated precipitation of the cationic component. The subject matter of the invention is also a method for the purification of polyhexamethylene biguanide, characterized in that polyhexamethylene biguanide hydrochloride (Structural Formula 1) precipitates out in aqueous solution with alkali hydrogen carbonate, the precipitate is separated from the mother liquor, and is converted back with hydrochloric acid into purified product according to Structural Formula 1. In this way, novel salts of the polyhexamethylene biguanide are obtained through the reaction of the polyhexamethylene biguanide hydrogen carbonate with inorganic or organic acids.

A use according to the invention is an antimicrobial material of absorbent wound dressings, characterized in that the material is realized with an insoluble salt of the hexamethylene biguanide by dip coating and/or spraying, wherein a concentration is maintained in which no significant diffusion of the biguanide into the wound takes place.

The invention will be explained in greater detail with reference to structural formulas and examples, without limiting the invention to these examples.

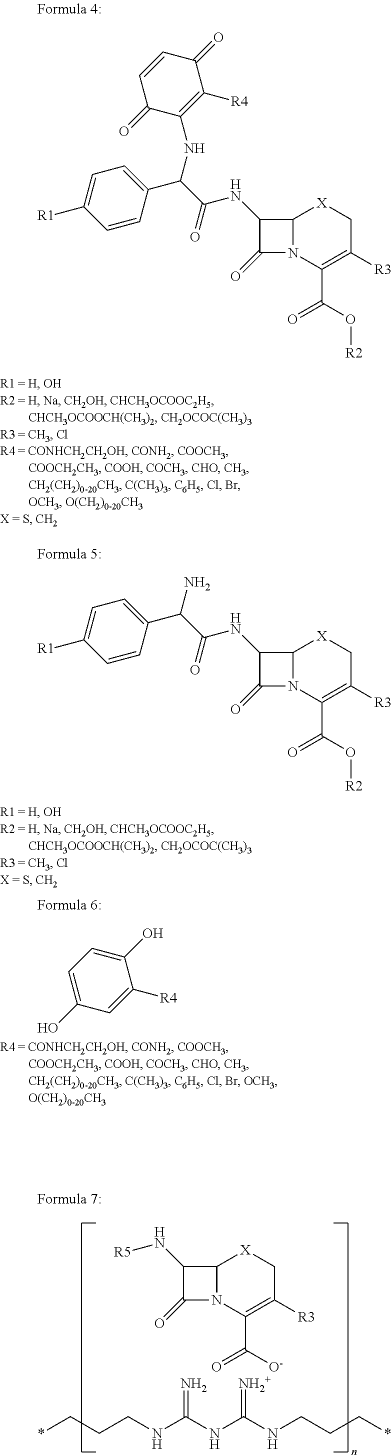

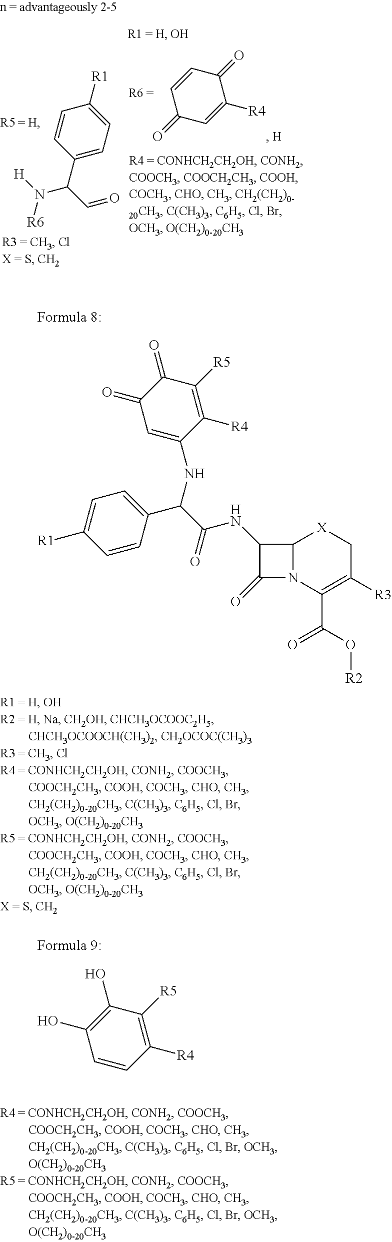

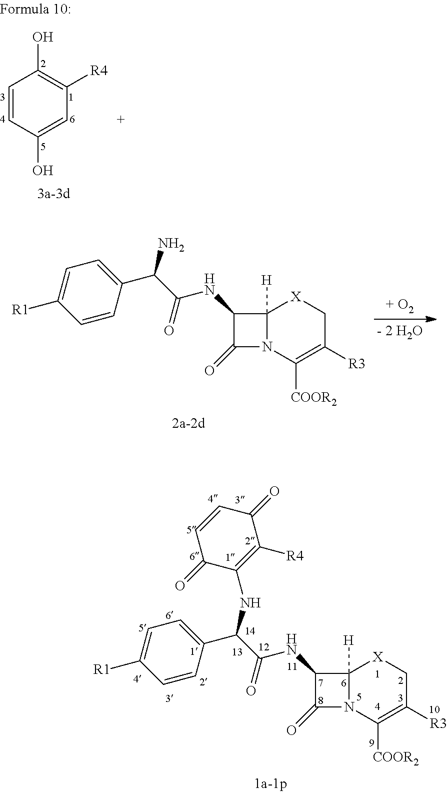

| | R1 | R2 | R3 | R4 | X |
|---|---|---|---|---|---|
| 1a | OH | H | CH₃ | | S |
| 1e | H | H | CH₃ | 7″ 8″ 9″ 10″ | S |
| 1i | H | H | Cl | CONHCH₂CH₂OH | S |
| 1m | H | H | Cl | | CH₂ |
| 1b | OH | H | CH₃ | | S |
| 1f | H | H | CH₃ | 7″ 8″ | S |
| 1j | H | H | Cl | CONH₂ | S |
| 1n | H | H | Cl | | CH₂ |
| 1c | OH | H | CH₃ | | S |
| 1g | H | H | CH₃ | 7″ 8″ | S |
| 1k | H | H | Cl | COOCH₃ | S |
| 1o | H | H | Cl | | CH₂ |
| 1d | OH | H | CH₃ | | S |
| 1h | H | H | CH₃ | 7″ 8″ 9″ | S |
| 1l | H | H | Cl | COOCH₂CH₃ | S |
| 1p | H | H | Cl | | CH₂ |

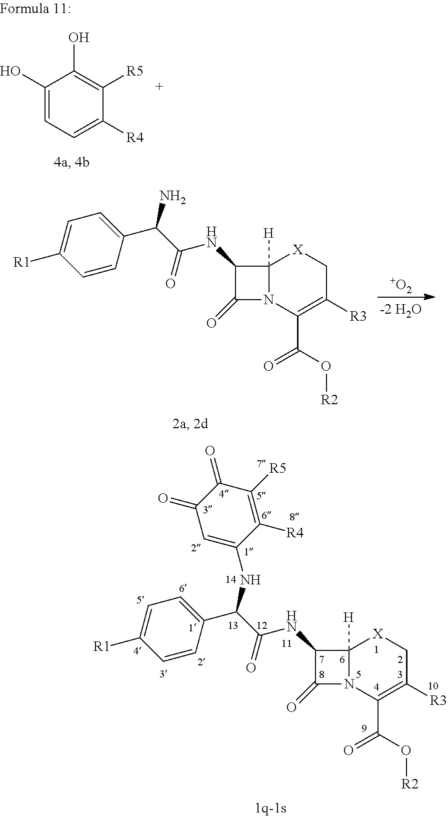

| | R1 | R2 | R3 | R4 | R5 | X |
|---|---|---|---|---|---|---|
| 1q | OH | H | CH₃ | H | CH₃ | S |
| 1r | OH | H | CH₃ | CH₃ | H | S |
| 1s | H | H | Cl | H | CH₃ | CH₂ |

### EXAMPLES

### General

The structural analyses and the tests on biological activity of Examples 1-36 form the basis of the designations of the structural elements and also the substances corresponding to Formula 10.

### Example 1

### 7-{2-2-(2-hydroxyethylcarbamoyl)-3,6-dioxyocyclohexa-1,4-dienylamino]-2-(4-hydroxyphenyl)-acetylamino}-desacetoxycephalosporanic acid 1a

The active ingredient was obtained through the reaction of active ingredients of the general Formula 5 (R1=OH, R2=H, R3=CH₃, X═S) with substances of the general Formula 6 (R4=CONHCH₂CH₂OH).

### Structural Analysis:

NMR spectra were recorded at 300 MHz (¹H) and 75 MHz (¹³C and DEPT-135) in acetonitrile-d₃. ¹H NMR δ 2.01 (s, 3H, H-10), 3.19 (d, J=18.3 Hz, 1H, H-2), 3.37 (m, J=5.7 Hz, 2H, H-9″), 3.43 (d, J=18.3 Hz, 1H, H-2), 3.57 (t, J=5.5 Hz, 2H, H-10″), 4.89 (d, J=4.7 Hz, 1H, H-6), 5.65 (dd, J=4.7 Hz, J=8.9 Hz, 1H, H-7), 5.87 (d, J=6.5 Hz, 1H, H-13), 6.56 (d, J=10.2 Hz, 1H, H-4″), 6.67 (d, J=10.1 Hz, 1H, H-5″), 6.82 (d, J=8.7 Hz, 2H, H-3′, H-5′), 7.39 (d, J=8.7 Hz, 2H, H-2′, H-6′), 7.52 (d, J=8.6 Hz, 1H, H-11), 9.77 (t, 1H, H-8″), 13.12 (d, 1H, J=6.2 Hz, H-14). ¹³C NMR δ 19.91 (C-10), 30.34 (C-2), 42.10 (C-9″), 57.97 (C-6), 59.82 (C-7), 61.52 (C-10″), 62.95 (C-13), 101.39 (C-1″), 116.65 (C-3′, C-5′), 123.15 (C-4), 129.83 (C-1′), 129.96 (C-2′, C-6′), 132.41 (C-3), 133.33 (C-4″), 141.34 (C-5″), 153.46 (C-2″), 158.46 (C-4′), 164.01 (C-8), 165.05 (C-9), 170.12 (C7″), 171.96 (C-12), 184.60 (C-6″), 185.52 (C-3″). LC/MS m/z 557.5 ([M+H]⁺, 597.5 [M+Na]⁺ API-ES pos. mode).

### Proof of Stability

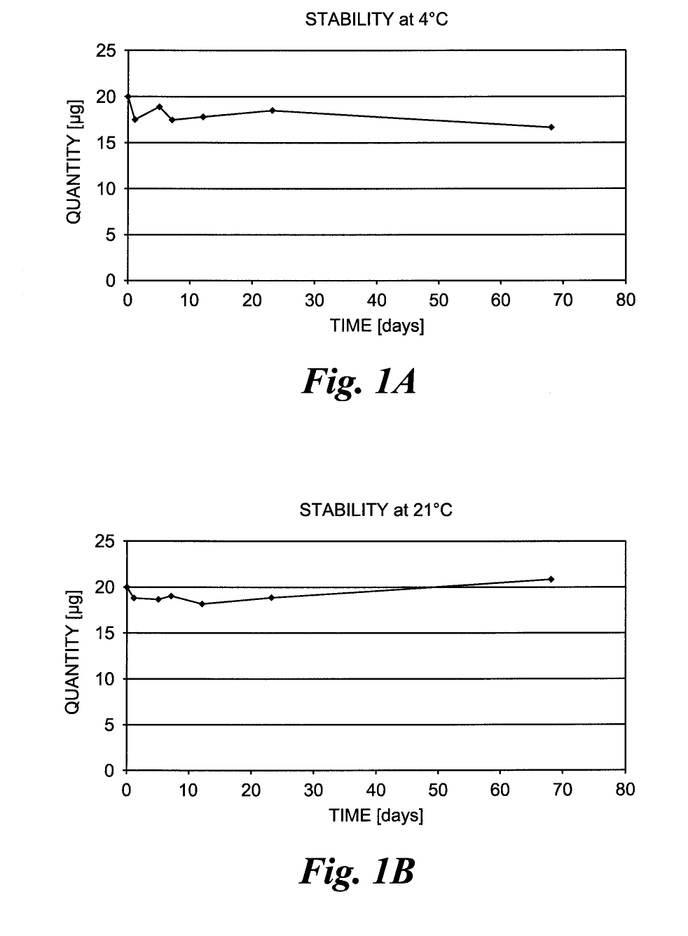

The novel active ingredient proved stable during storage over a time period >60 days. FIG. 1 shows the storage stability of 7-{2-[2-(2-hydroxyethylcarbamoyl)-3,6-dioxocyclohexa-1,4-dienylamino]-2-(4-hydroxyphenyl)-acetylamino}-desacetoxycephalosporanic acid 1a.

### Example 2

### 7-[2-(2-carbamoyl-3,6-dioxocyclohexa-1,4-dienylamino)-2-(4-hydroxyphenyl)-acetylamino]-desacetoxycephalosporanic acid 1b

The active ingredient was obtained by the reaction of active ingredients of the general Formula 5 (R1=OH, R2=H, R3=CH₃, X═S) with substances of the general Formula 6 (R4=CONH₂).

### Structural Analysis

NMR spectra were recorded at 300 MHz (¹H) in acetonitrile-d₃.

¹H NMR δ 2.01 (s, 3H, H-10), 3.15 (d, J=18.4, 1H, H-2), 3.44 (d, J=18.4 Hz, 1H, H-2), 4.89 (d, J=4.7 Hz, 1H, H-6), 5.65 (dd, J=4.7 Hz, J=8.8 Hz, 1H, H-7), 5.89 (d, J=6.9 Hz, 1H, H-13), 6.58 (d, J=10.3 Hz, 1H, H-4″), 6.69 (d, J=10.3 Hz, 1H, H-5″), 6.82 (d, J=8.7 Hz, 2H, H-3′, H-5′), 7.30 (d, J=8.6 Hz, 2H, H-2′, H-6′), 7.39 (d, J=8.7 Hz, 1H, H-11), 9.07 (s, 2H, H-8″), 13.13 (d, 1H, J=6.8 Hz, H-14). LC/MS m/z 515.1 ([M+H]⁺, 535.1 [M+Na]⁺ API-ES pos. mode).

### Example 3

### 7-[2-(4-hydroxyphenyl)-2-(2-methoxycarbonyl-3,6-dioxocyclohexa-1,4-dienylamino)-acetylamino]-desacetoxycephalosporanic acid 1c

The active ingredient was obtained through the reaction of active ingredients of the general Formula 5 (R1=OH, R2=H, R3=CH₃, X═S) with substances of the general Formula 6 (R4=COOCH₃).

### Structural Analysis:

NMR spectra were recorded at 300 MHz (¹H) in acetonitrile-d₃.

¹H NMR δ 1.99 (s, 3H, H-10), 3.12 (d, J=18.1 Hz, 1H, H-2), 3.42 (d, J=18.1 Hz, 1H, H-2), 3.64 (br s, 3H, H-8″), 4.86 (d, J=4.6 Hz, 1H, H-6), 5.63 (dd, J=4.6 Hz, J=8.8 Hz, 1H, H-7), 5.89 (d, J=6.9 Hz, 1H, H-13), 6.58 (d, J=10.0 Hz, 1H, H-4″), 6.67 (d, J=10.1 Hz, 1H, H-5″), 6.77 (d, J=8.5 Hz, 2H, H-3′, H-5′), 7.20 (d, J=8.5 Hz, 2H, H-2′, H-6′), 7.52 (d, J=8.7 Hz, 1H, H-11), 13.17 (d, 1H, J=6.8 Hz, H-14). LC/MS m/z 528.3 ([M+H]⁺, 550.1 [M+Na]⁺ API-ES pos. mode).

### Example 4

### 7-[2-(2-ethoxycarbonyl-3,6-dioxocyclohexa-1,4-dienylamino)-2-(4-hydroxyphenyl)-acetylamino]-desacetoxycephalosporanic acid 1d

The active ingredient was obtained through the reaction of active ingredients of the general Formula 5 (R1=OH, R2=H, R3=CH₃, X═S) with substances of the general Formula 6 (R4=COOCH₂CH₃).

### Structural Analysis:

NMR spectra were recorded at 300 MHz (1H) in acetonitrile-d₃.

¹H NMR δ 1.14 (br, 3H, H-9″), 2.01 (s, 3H, H-10), 3.14 (d, J=18.4 Hz, 1H, H-2), 3.43 (d, J=18.4 Hz, 1H, H-2), 4.08 (br, 2H, H-8″), 4.87 (d, J=4.7 Hz, 1H, H-6), 5.66 (dd, J=4.7 Hz, J=8.8 Hz, 1H, H-7), 5.89 (d, J=6.9 Hz, 1H, H-13), 6.58 (d, J=10.2 Hz, 1H, H-4″), 6.68 (d, J=10.2 Hz, 1H, H-5″), 6.78 (d, J=8.5 Hz, 2H, H-3′, H-5′), 7.20 (d, J=8.4 Hz, 2H, H-2′, H-6′), 7.55 (d, J=8.7 Hz, 1H, H-11), 13.17 (d, 1H, J=6.7 Hz, H-14). LC/MS m/z 543.9 ([M+H]⁺, 564.0 [M+Na]⁺ API-ES pos. mode).

### Example 5

Antimicrobial effect of the novel compounds according to Formula 4 in vitro

### Methods:

### Test System 1 (Antimicrobial Effect)

### Pre-Culture:

The test germs Escherichia coli SBUG 1135 and Bacillus megaterium SBUG 1152 are drawn overnight (17 hours at 37° C.) in 5 ml nutrient medium II. The incubation is performed in a shaking incubator (INFORS AG CH 4103, Bottmingen, Switzerland) at a shaking frequency of 180 rpm.

After 17 h incubation time, Escherichia coli has reached a cell density of ca. 2.4×10¹⁰ cells/ml and Bacillus megaterium a cell density of ca. 2.1×10⁸ cells/ml.

### Inoculation of Nutrient Agar:

The seeding of bacteria in agar is selected so that, dense, but not confluent individual colonies develop after the incubation.

The following quantities are used:

Bacillus megaterium: 0.1 ml of non-diluted overnight culture (ÜN) in 10 ml nutrient agar corresponds to a cell count of 10⁶.

Escherichia coli: the ÜN is diluted 1:100 with physiological saline solution, of which 0.1 ml is converted into 10 ml nutrient agar. That corresponds to a cell count of 10⁶.

The inoculated nutrient agar is set in sterile Petri dishes and left standing for a few minutes for drying.

### Test for Antimicrobial Effect:

The test substances are deposited in stepped quantities (10 μg, 50 μg, 100 μg) onto active ingredient carriers (sensi-discs). For this purpose, the test substances are dissolved in methanol or A. dest. according to their solubility. Each inoculated plate is fitted with 3 test sheets.

After a 20-hour incubation time at 37° C., the zones of inhibition around the individual test sheets could be measured. The diameter of the zone of inhibition is specified in mm.

### Test System II (Effect Against Multi-Resistant Germs)

For this purpose, bacteria is cultivated on Müller-Hinton-Agar II plates.

With 1-1.5 ml physiological saline solution, a bacteria suspension of McFarland 0.5 (corresponds to a bacteria density of 150×10⁶ germs) is produced. Then, with a sterile glass rod, a small drop of bacteria suspension is placed on the Müller-Hinton-Agar plate and coated in three layers (vertical, horizontal, and transverse). Thereafter, the sensi-discs with the novel semi-synthetic test substances are placed on top. The fitted plates are incubated for 18-20 hours at 37° C. After incubation, the diameters of the zones of inhibition are read as a measure for the antimicrobial activity of the novel semi-synthetic test substances.

Results: The novel active ingredients have a strong antimicrobial effect (Table 1).

### Example 6

### Adaptation of the Cationic Component of the Active Ingredient According to Formula 2 to Biological Requirements, e.g., Antimicrobial Effect Through Fractionated Precipitation

Through fractionated precipitation of commercially available polyhexanide (Dr. Trippen GmbH, Freiburg) with sodium hydrogen carbonate, the polymer mixture was split into individual fractions that differ from each other significantly in their solubility and in their lipid-water distribution relationship.

### Method According to the Invention

Antimicrobial effectiveness of active ingredients of the general Formula 2 in which amoxicillin represents the anion.

Methods cf. Example 5
Results: The novel active ingredient exceeds the effectiveness of the commercial product Lavasept® (Desomed AG) considerably (Table 2).

TABLE 1
Antimicrobial effect of the novel active ingredients 1a to 1d and 2a.

| | Quantity n [mol] | | | | | | | | | | | | | | |
|---|---|---|---|---|---|---|---|---|---|---|---|---|---|---|---|
| | 2a | | | 1a | | | 1b | | | 1c | | | 1d | | |
| Strain | 0.019 | 0.1 | 0.19 | 0.019 | 0.1 | 0.19 | 0.019 | 0.1 | 0.19 | 0.019 | 0.1 | 0.19 | 0.029 | 0.14 | 0.29 |
| Bacillus | 36¹ | 38 | 40 | 22 | 30 | 32 | 22 | 30 | 32 | 22 | 30 | 32 | 18 | 30 | 34 |
| megaterium | | | | | | | | | | | | | | | |
| SBUG 1152 | | | | | | | | | | | | | | | |
| B. subtilis | 36 | 38 | 40 | 24 | 30 | 32 | 22 | 30 | 32 | 22 | 30 | 32 | 22 | 30 | 34 |
| AWD 166 | | | | | | | | | | | | | | | |
| Escherichia coli | 18 | 22 | 26 | r² | 14 | 16 | r | 14 | 18 | r | 14 | 18 | r | 12 | 16 |
| SBUG 1135 | | | | | | | | | | | | | | | |
| Enterococcus | r | 10 | 14 | r | r | r | r | r | r | r | r | r | r | r | r |
| faecalis 769 | | | | | | | | | | | | | | | |
| E. faecalis 945 | r | r | r | r | r | r | r | r | r | r | r | r | r | r | r |
| Staphylococcus | r | r | 8 | r | 10 | 16 | r | r | 8 | r | r | 12 | r | r | 8 |
| aureus 315 | | | | | | | | | | | | | | | |
| S. aureus 33490 | 16 | 24 | 28 | r | 14 | 18 | r | 14 | 20 | r | 14 | 20 | r | 14 | 16 |
| S. aureus 34289 | r | r | r | r | r | r | r | r | 12 | r | r | r | r | r | r |
| S. aureus 36881 | r | 14 | 18 | r | 10 | 16 | r | 12 | 18 | r | r | 14 | r | 14 | 18 |
| S. aureus 38418 | 30 | 34 | 38 | 14 | 24 | 26 | 16 | 24 | 26 | 10 | 22 | 28 | 10 | 20 | 24 |
| S. aureus 39105 | 16 | 24 | 26 | r | r | 14 | r | 12 | 18 | r | r | 12 | r | r | 14 |
| S. aureus 520 | r | r | 8 | r | r | 12 | r | r | r | r | r | r | r | r | r |
| S. aureus 526 | r | r | r | r | r | 10 | r | r | 12 | r | r | r | r | r | r |
| S. aureus | 32 | 38 | 40 | 20 | 28 | 30 | 16 | 24 | 28 | 18 | 26 | 30 | 14 | 22 | 26 |
| ATCC 6538 | | | | | | | | | | | | | | | |
| S. aureus North | r | r | 10 | r | 16 | 18 | r | 8 | 12 | r | 10 | 14 | r | 10 | 12 |
| Germany strain | | | | | | | | | | | | | | | |
| S. epidermidis | 20 | 24 | 26 | r | 10 | 16 | r | 16 | 20 | r | 14 | 20 | r | 12 | 14 |
| 1068 | | | | | | | | | | | | | | | |
| S. epidermidis | 22 | 28 | 30 | r | 18 | 20 | r | 18 | 22 | r | 14 | 20 | r | 14 | 18 |
| 1071 | | | | | | | | | | | | | | | |
| S. epidermidis | 26 | 36 | 40 | 14 | 24 | 28 | 14 | 22 | 26 | 12 | 24 | 28 | 10 | 20 | 24 |
| 125 | | | | | | | | | | | | | | | |
| S. epidermidis | 24 | 28 | 32 | r | 10 | 16 | r | 14 | 20 | r | 14 | 20 | r | 12 | 18 |
| 563 | | | | | | | | | | | | | | | |
| S. epidermidis | 12 | 26 | 30 | r | 16 | 20 | r | 10 | 12 | r | 14 | 18 | r | 10 | 14 |
| 847 | | | | | | | | | | | | | | | |

¹Diameter of zone of inhibition in mm
²r = resistant (no detectable zone of inhibition)

TABLE 2
Antimicrobial effect of compounds according to Formula
2 with X⁻ = amoxicillin anion against multi-resistant
bacteria in comparison with Lavasept (quantity 0.09 μmol)

| | Diameter of zone of inhibition | |
|---|---|---|
| | | Polyhexamethylene biguanid |
| Test germ | Lavasept | cation + amoxicillin anion |
| Staphylococcus aureus ATCC 6538 | 12 mm¹ | 44 mm |
| Staphylococcus aureus 33490 | 10 mm | 34 mm |
| Staphylococcus aureus 36881 | 10 mm | 20 mm |
| Staphylococcus aureus 38418 | 10 mm | 40 mm |
| Staphylococcus aureus 520 | r | 14 mm |
| Staphylococcus aureus 315 | r | 18 mm |
| Staphylococcus aureus col. | 12 mm | 34 mm |
| Staphylococcus epidermidis 125 | 10 mm | 32 mm |
| Staphylococcus epidermidis 847 | 10 mm | 32 mm |
| Staphylococcus haemolyticus 535 | 10 mm | 16 mm |
| Enterococcus faecalis 769 | r | 36 mm |

¹Diameter of zone of inhibition in mm,
²r = resistant (no detectable zone of inhibition)

TABLE 3
Antimicrobial effect of compounds according to Formula 3 with X⁻ = 6-{2-[2-(2-hydroxyethylcarbamoyl)-
3,6-dioxocyclohexa-1,4-dienylamino]-2-(4-hydroxyphenyl)-acetylamino}-penicillanate against
multi-resistant bacteria in comparison with 6-{2-[2-(2-hydroxyethylcarbamoyl)-3,6-dioxyocyclohexa-
1,4-dienylamino]-2-(4-hydroxyphenyl)-acetylamino}-penicillanic acid (quantity 0.09 μmol)

| | Diameter of zone of inhibition | | |
|---|---|---|---|
| | | 6-{2-[2-(2- | |
| | | hydroxyethylcarbamoyl)- | Active ingredient according to |
| | | 3,6-dioxocyclohexa-1,4- | Formula 2 with X⁻ = 6-{2-[2-(2- |
| | | dienylamino]-2-(4- | hydroxyethylcarbamoyl)-3,6- |
| | | hydroxyphenyl)- | dioxocyclohexa-1,4-dienylamino]-2- |
| | | acetylamino- | (4-hydroxyphenyl)-acetylamino}- |
| Test germ | Lavasept | penicillanic acid | penicillanate |
| Staphylococcus aureus ATCC 6538 | 12 mm¹ | 44 mm | 42 mm |
| Staphylococcus aureus 33490 | 10 mm | 18 mm | 34 mm |
| Staphylococcus aureus 36881 | 10 mm | r² | 18 mm |
| Staphylococcus aureus 38418 | 10 mm | 34 mm | 40 mm |
| Staphylococcus aureus 520 | r | r | 14 mm |
| Staphylococcus aureus 315 | r | r | 18 mm |
| Staphylococcus aureus col. | 12 mm | 32 mm | 30 mm |
| Staphylococcus epidermidis 125 | 10 mm | 28 mm | 30 mm |
| Staphylococcus epidermidis 847 | 10 mm | 24 mm | 28 mm |
| Staphylococcus haemolyticus 535 | 10 mm | r | 14 mm |
| Enterococcus faecalis 769 | r | 28 mm | 34 mm |

¹r = resistant (no detectable zone of inhibition)

The novel active ingredient causes inhibition in all of the tested multi-resistant bacteria strains, even in strains in which both the cation and also the antibiotic without a cationic component are ineffective (Table 3).

### Example 7

### Production of Novel Cephalosporins Through the Formation of Salts with Cations that Themselves have an Active Ingredient Character

The fractions according to Example 6 were converted with 7-aminocephalosporanic acid and also with active ingredients according to Formula 4 (R1=OH, R2=H, R3=CH₃, R4=CONHCH₂CH₂OH, X═S) and with active ingredients according to Formula 5 (R1=OH, R2=H, R3=CH₃, X═S). Here, products with strong antimicrobial effects were obtained.

### Example 8

### Antimicrobial Effect In Vitro of the Substances According to Formula 7 (Example 7)

### Method

A series of dilution tests was performed. The concentration was determined in weight percent that causes an absolute inhibition in growth (minimal inhibition concentration, MHK).

### Results

The active ingredients according to Formula 7 are effective against a wide spectrum of germs (Table 4).

TABLE 4
MHK of active ingredients of the general Formula 7 in μmol/l

| | Staphylococcus | Bacillus | Proteus | | Serratia |
|---|---|---|---|---|---|
| | aureus | subtili | mirabilis | E. coli | marcescens |
| | SBUG 11 | SBUG 14s | SBUG 47 | SBUG 17 | SBUG 9 |
| Active ingredient | 0.06 | 0.06 | 0.12 | 0.5 | 0.5 |
| according to | | | | | |
| Formula 7 | | | | | |

### Example 9

### Proof of the Antimicrobial Effect of the Compounds According to Example 6 in Local Application

### Method

The proof was performed using the “mouse ear test” model:

After killing the animals, the mouse ears were sectioned and mounted in a special holding device. The contamination of the ears was performed with 5 μl of a 1:10 diluted suspension of the MRSA strain “North German epidemic strain” with an optical density according to the McFarland standard 0.5. After incubation of 1.5 h at 30° C., half of the ears are treated with the active ingredients according to the invention from Example 6 and the other half is left untreated. The growing bacteria colonies are evaluated after 24 h incubation at 37° C.

### Results

In the untreated ears, germ counts between 1000 and 10,000 were observed. In contrast, for the studied active ingredients according to Example 6, the germ growth was completely inhibited.

### Example 10

### The Working of Active Ingredients into Lipids and Production of Micro-Particles and Nano-Particles

### Method

### Formulation

| Active ingredient | |
|---|---|
| according to the | Quantity |
| invention | in g |
| Active ingredient | 0.1 |
| according to Example 1 | |
| Lipid mixture | 10.00 |
| Emulsifying agent | 0.1 |
| (Plantacare 2000) | |
| Demineralized water | ad 100.00 |

The lipids are heated to a temperature of 50° C. and then the active ingredient being used according to Example 1 is dispersed therein. Apart from this, an aqueous emulsifying agent solution is heated to the corresponding temperature (50° C.). Thereafter, both phases are combined at the desired homogenization temperature. Then, the mixture is processed with the help of an Ultra Turax T25 from Janke and Kunkel GmbH & Co. KG (Staufen, Germany) in an emulsification process at 8000 revolutions per minute and a period of 30 seconds.

The suspension is then homogenized four times with a piston-gap high-pressure homogenizer Micron Lab 40 (APV-Gaulin, Lubeck) at a pressure of 500 bar and a temperature of 50° C. The resulting formulation was tested as described in Example 9 for its antimicrobial effectiveness for use on skin.

### Result

Germ growth on the treated skin was completely inhibited.

### Example 11

### Antimicrobial Effectiveness In Vitro of the Novel Substances According to Formula 4 (Example 1-Example 4)

Method for Effectiveness In Vivo with the Mouse Infected with Staphylococcus aureus Model

### Pre-Treatment of the Mice:

On Day 0, at least 3 BALB/c mice per test substance (Table 8) or controls are pretreated with cyclophosphamide (250 mg/kg) in 250 μl PBS inta peritoneal (i.p.).

On Day 2, the animals again received 100 mg/kg i.p.

### Bacteria Pre-Culture:

On Day 2, a colony of the test germ is transferred from a growing agar plate (Müller-Hinton-Agar, Beckton Dickinson) into a flask with 10 ml CASO broth (CASO-B., Soyabean peptone-Casein peptone broth, SIFIN, 30 g/l) and is cultivated overnight at 37° C. and a shaking frequency of 250 rpm.

On Day 3, the pre-culture (Vk) is diluted 1:50 with CASO-B and further cultivated ca. 2 hours until an extinction in the medium of 0.6 at a wavelength of 550 nm is reached. Then, it is grown 1× with PBS at room temperature and set again to an extinction of 0.6.

### Infection:

At least 3 animals for a test substance or controls are infected with 10 μl/g body weight grown germs of a bacteria suspension with the extinction 0.6 (ca. 10¹⁰-10¹² CFU, Colony forming units) i.p.

### Test Substances:

### Variant 1:

After 30 minutes, antibiotic solution is injected into the mice in a volume of 200 μl PBS with 3% DMSO i.p. 6 hours later, the second antibiotic injection is performed at the same concentration.

### Variant 2:

After 6 and 20 hours, antibiotic solution is injected into the mice in a volume of 200 μl PBS with 3% DMSO i.p.

### Result

In the “mouse infected with Staphylococcus aureus” infection model, the animals without effective treatment die at 100%. After treatment with the active ingredient according to the invention from Formula 4, all of the animals survive (Table 5).

TABLE 5
Effectiveness of in vitro active ingredients in the “mouse infected
with Staphylococcus aureus” infection model

| | | Survival/ | Survival/ |
|---|---|---|---|
| Active ingredient | Tested | tested | controls |
| according to the | concentrations | mice n/n | n/n |
| invention | Staphylococcus aureus ATCC 6538 | | |
| 2a | 2 × 0.5 mg | 10/10 | 0/10 |
| | (25 mg/kg) | | |
| 1a | 2 × 0.5 mg | 9/9 | 0/13 |
| | (25 mg/kg) | | |
| 1b | 2 × 1.0 mg | 6/6 | 0/10 |
| | (50 mg/kg) | | |
| 1d | 2 × 0.5 mg | 4/6 | 2/10 |
| | (25 mg/kg) | | |

### Example 12

### Targeted Increase in the Lipophilicity of the Commercially Available Antibiotic Cefadroxil (Formula 5, R1=OH, R2=H, R3=CH₃, X═S) Through the Radical-Mediated Introduction of Novel Substitutes

In order to achieve sufficient fixing on a lipophilic surface, the lipophilicity of the commercially available antibiotic Cefadroxil (Formula 5, R1=OH, R2=H, R3=CH₃, X═S) shall be increased selectively through the radical-mediated introduction of paradihydroxylated substitutes. Initially, the distribution behavior of Cefadroxil was set between an aqueous and a lipophilic phase according to the following method.

### Method

Setting the distribution behavior by determining the logP value according to the method by Donovan et al., (Journal of Chromatography A, 952:47-61, 2002,) by means of HPLC.

### Chromatography Conditions:

Operating temperature 18-24° C.
Flow 1.5 mL/min
Linear gradient (methanol/0.1% phosphoric acid pH 2) from 10% to 100% methanol in 9.4 min, equilibrium time between 2 runs 6 min

### Sample Preparation:

ca. 0.5 mg of analyte is added to 0.5 ml of a standard solution (440 mg methyl phenyl sulfonate and 380 μl toluene in 150 mL methanol). 2 μl of this sample solution is injected.

### Calculation:

Because the distribution coefficients of the two standards running in each test are known, the distribution coefficient of the analytes can be determined from the relationships of the analyte/standard retention times.

### Result:

The determined logP value of the commercially available antibiotic Cefadroxil (Formula 5, R1=OH, R2=H, R3=CH₃, X═S) equals −3.50. In order to achieve sufficient bonding to a lipophilic bearing matrix, the lipophilicity should be increased selectively.

The problem was solved according to the invention by radical-mediated reactions corresponding to Example 1 with 2,5-dihydroxy-N-(2-hydroxyethyl)benzamide (logP value=−1.21; Formula 6, R4=CONHCH₂CH₂OH), corresponding to Example 2 with 2,5-dihydroxybenzamide (logP value=−0.4; Formula 6, R4=CONH₂), and corresponding to Example 4 with 2,5-dihydroxybenzoic acid ethylester (logP value=2.65; Formula 6, R4=COOCH₂CH₃).

Antibiotics with significantly higher lipophilicity (Table 6) were obtained while maintaining the antimicrobial activity (Table 1).

TABLE 6
logP values

| Active ingredient | |
|---|---|
| according to the | |
| invention | logP value |
| 1a | 1.00 |
| 1b | 1.33 |
| 1d | 1.76 |

### Example 13

### 7-{2-[2-(2-hydroxyethylcarbamoyl)-3,6-dioxocyclohexa-1,4-dienylamino]-2-phenyl-acetylamino}-desacetoxycephalosporanic acid 1e

The active ingredient was obtained through the reaction of active ingredients of the general Formula 5 (R1=H, R2=H, R3=CH₃, X═S) with substances of the general Formula 6 (R4=CONHCH₂CH₂OH).

### Structural Analysis

NMR spectra were recorded at 300 MHz (¹H) and 75 MHz (¹³C and DEPT-135) in acetonitrile-d₃. ¹H NMR δ 2.01 (s, 3H, H-10), 3.15 (d, J=18.4 Hz, 1H, H-2), 3.37 (m, J=5.7 Hz, 2H, H-9″), 3.44 (d, J=18.4 Hz, 1H, H-2), 3.57 (t, J=5.6 Hz, 2H, H-10″), 4.90 (d, J=4.7 Hz, 1H, H-6), 5.69 (dd, J=4.6 Hz, J=8.9 Hz, 1H, H-7), 6.00 (d, J=6.5 Hz, 1H, H-13), 6.59 (d, J=10.2 Hz, 1H, H-4″), 6.71 (d, J=10.1 Hz, 1H, H-5″), 7.46 (m, 6H, H-2′, H-3′, H-4′, H-5′, H-6′, H-11), 9.79 (t, 1H, H-8″), 13.29 (d, 1H, J=6.2 Hz, H-14). ¹³C NMR δ 19.93 (C-10), 30.35 (C-2), 42.10 (C-9″), 57.95 (C-6), 59.81 (C-7), 61.50 (C-10″), 63.15 (C-13), 101.42 (C-1″), 123.15 (C-4), 128.41 (C-2′, C-6′), 129.84 (C-4′), 130.09 (C-3′, C-5′), 132.41 (C-3), 133.33 (C-4″), 138.36 (C-1′), 141.34 (C-5″), 153.39 (C-2″), 161.46 (C-8), 165.01 (C-9), 170.12 (C7″), 171.96 (C-12), 184.60 (C-6″), 185.52 (C-3″). LC/MS m/z 543.1 ([M+H]⁺, 563.0 [M+Na]⁺ API-ES pos. mode).

### Example 14

### 7-[2-(2-carbamoyl-3,6-dioxocyclohexa-1,4-dienylamino)-2-phenyl-acetylamino]-desacetoxycephalosporanic acid 1f The active ingredient was obtained through the reaction of active ingredients of the general Formula 5 (R1=H, R2=H, R3=CH₃, X═S) with substances of the general Formula 6 (R4=CONH₂).

### Structural Analysis

NMR spectra were recorded at 300 MHz (1H) in acetonitrile-d₃.

¹H NMR δ 2.02 (s, 3H, H-10), 3.15 (d, J=18.4 Hz, 1H, H-2), 3.44 (d, J=18.4 Hz, 1H, H-2), 4.90 (d, J=4.6 Hz, 1H, H-6), 5.69 (dd, J=4.5 Hz, J=8.5 Hz, 1H, H-7), 6.00 (d, J=6.7 Hz, 1H, H-13), 6.59 (d, J=10.2 Hz, 1H, H-4″), 6.71 (d, J=10.2 Hz, 1H, H-5″), 7.46 (m, 5H, H-2′, H-3′, H-4′, H-5′, H-6′), 7.59 (d, J=8.4 Hz, 1H, H-11), 9.09 (s, 2H, H-8″), 13.29 (d, 1H, J=6.7 Hz, H-14). LC/MS m/z 497.0 ([M+H]⁺, 519.0 [M+Na]⁺ API-ES pos. mode).

### Example 15

### 7-[2-(2-methoxycarbonyl-3,6-dioxocyclohexa-1,4-dienylamino)-2-phenyl-acetylamino]-desacetoxycephalosporanic acid 1g

The active ingredient was obtained through the reaction of active ingredients of the general Formula 5 (R1=H, R2=H, R3=CH₃, X═S) with substances of the general Formula 6 (R4=COOCH₃).

### Structural Analysis

NMR spectra were recorded at 300 MHz (1H) in acetonitrile-d₃.

¹H NMR δ 2.01 (s, 3H, H-10), 3.14 (d, J=18.2 Hz, 1H, H-2), 3.43 (d, J=18.2 Hz, 1H, H-2), 3.65 (br s, 3H, H-8″), 4.88 (d, J=4.6 Hz, 1H, H-6), 5.65 (dd, J=4.6 Hz, J=8.8 Hz, 1H, H-7), 5.92 (d, J=6.8 Hz, 1H, H-13), 6.57 (d, J=10.2 Hz, 1H, H-4″), 6.69 (d, J=10.1 Hz, 1H, H-5″), 7.47 (m, 5H, H-2′, H-3′, H-4′, H-5′, H-6′), 7.52 (d, J=8.8 Hz, 1H, H-11), 13.18 (d, 1H, J=6.8 Hz, H-14). LC/MS m/z 510.0 ([M+H]⁻ API-ES neg. mode), 534.0 ([M+Na]⁺ API-ES pos. mode).

### Example 16

### 7-[2-(2-ethoxycarbonyl-3,6-dioxocyclohexa-1,4-dienylamino)-2-phenyl-acetylamino]-desacetoxycephalosporanic acid 1h

The active ingredient was obtained through the reaction of active ingredients of the general Formula 5 (R1=H, R2=H, R3=CH₃, X═S) with substances of the general Formula 6 (R4=COOCH₂CH₃).

### Structural Analysis

NMR spectra were recorded at 300 MHz (1H) in acetonitrile-d₃.

¹H NMR δ 1.12 (br, 3H, H-9″), 2.01 (s, 3H, H-10), 3.12 (d, J=18.2 Hz, 1H, H-2), 3.40 (d, J=18.2 Hz, 1H, H-2), 3.99 (br, 2H, H-8″), 4.86 (d, J=4.7 Hz, 1H, H-6), 5.67 (dd, J=4.6 Hz, J=8.7 Hz, 1H, H-7), 6.19 (d, J=8.4 Hz, 1H, H-13), 6.58 (d, J=10.1 Hz, 1H, H-4″), 6.68 (d, J=10.2 Hz, 1H, H-5″), 7.37 (m, 5H, H-2′, H-3′, H-4′, H-5′, H-6′), 7.46 (d, J=8.7 Hz, 1H, H-11). LC/MS m/z 426.3 ([M+H]⁺, 548.1 [M+Na]⁺ API-ES pos. mode).

### Example 17

### Antimicrobial Effect of the Novel Compounds According to Formula 4 (Example 13-Example 16) In Vitro

Methods cf. Example 5

### Results

The novel active ingredients have a strong antimicrobial effect (Table 7).

### Example 18

### Production of Novel Cephalosporins Through the Formation of Salts with Cations that Themselves have an Active Ingredient Character

The fractions according to Example 6 were reacted with active ingredients according to Formula 4 (R1=H, R2=H, R3=CH₃, R4=CONHCH₂CH₂OH, X═S) and with active ingredients according to Formula 5 (R1=H, R2=H, R3=CH₃, X═S). In this way, products with a strong antimicrobial effect were obtained.

### Example 19

### Antimicrobial Effectiveness In Vivo of the Novel Substances According to Formula 4 (Example 13, Example 14)

Methods cf. Example 11

### Result

In the “mouse infected with Staphylococcus aureus” model, the animals without effective treatment died at 100%. After treatment with the active ingredient according to the invention from Formula 3, all of the animals survived (Table 8).

TABLE 7
Antimicrobial effect of the novel active ingredients 1e to 1h and 2b.

| | Quantity n [mol] | | | | | | | | | | | | | | |
|---|---|---|---|---|---|---|---|---|---|---|---|---|---|---|---|
| | 2b | | | 1e | | | 1f | | | 1g | | | 1h | | |
| Strain | 0.019 | 0.1 | 0.19 | 0.029 | 0.14 | 0.29 | 0.02 | 0.1 | 0.2 | 0.029 | 0.14 | 0.29 | 0.019 | 0.09 | 0.19 |
| Bacillus | 32¹ | 38 | 40 | 20 | 28 | 32 | 20 | 30 | 32 | 20 | 30 | 34 | 18 | 28 | 32 |
| megaterium | | | | | | | | | | | | | | | |
| SBUG 1152 | | | | | | | | | | | | | | | |
| B. subtils | 30 | 36 | 38 | 22 | 30 | 32 | 24 | 30 | 32 | 24 | 30 | 32 | 22 | 30 | 32 |
| AWD 166 | | | | | | | | | | | | | | | |
| Escherichia | 20 | 26 | 30 | r² | 16 | 18 | r | 14 | 18 | r | 16 | 22 | r | 14 | 18 |
| coli SBUG | | | | | | | | | | | | | | | |
| 1135 | | | | | | | | | | | | | | | |
| Enterococcus | r | r | r | r | r | r | r | r | r | r | r | r | r | r | r |
| faecalis 769 | | | | | | | | | | | | | | | |
| E. faecalis | r | r | r | r | r | r | r | r | r | r | r | r | r | r | r |
| 945 | | | | | | | | | | | | | | | |
| Staphylococcus | r | 10 | 14 | r | r | 12 | r | r | 10 | r | r | 12 | r | r | 10 |
| aureus 315 | | | | | | | | | | | | | | | |
| S. aureus 33490 | 20 | 26 | 30 | 8 | 16 | 20 | r | 14 | 16 | r | 20 | 24 | r | 16 | 20 |
| S. aureus 34289 | r | r | r | r | r | r | r | r | r | r | r | r | r | r | r |
| S. aureus 36881 | r | 20 | 24 | r | r | 14 | r | r | 14 | r | r | 12 | r | r | 12 |
| S. aureus 38418 | 26 | 36 | 38 | 14 | 26 | 30 | 14 | 24 | 30 | 16 | 26 | 30 | 12 | 22 | 28 |
| S. aureus 39105 | 14 | 24 | 28 | r | r | 14 | r | r | 12 | r | 10 | 18 | r | 8 | 14 |
| S. aureus 520 | r | r | r | r | r | r | r | r | r | r | r | r | r | r | r |
| S. aureus 526 | r | r | r | r | r | r | r | r | r | r | r | r | r | r | r |
| S. aureus | 30 | 36 | 40 | 20 | 30 | 32 | 20 | 30 | 34 | 20 | 28 | 32 | 20 | 28 | 30 |
| ATCC 6538 | | | | | | | | | | | | | | | |
| S. aureus North | r | 8 | 12 | r | 10 | 16 | r | r | 10 | r | 10 | 14 | r | 8 | 12 |
| German strain | | | | | | | | | | | | | | | |
| S. epidermidis | 16 | 24 | 28 | r | 14 | 16 | r | 12 | 16 | r | 14 | 18 | r | 10 | 16 |
| 1068 | | | | | | | | | | | | | | | |
| S. epidermidis | 22 | 28 | 30 | 10 | 18 | 20 | 8 | 16 | 18 | r | 18 | 22 | r | 14 | 20 |
| 1071 | | | | | | | | | | | | | | | |
| S. epidermidis | 24 | 34 | 38 | r | 24 | 28 | 10 | 22 | 28 | 14 | 24 | 28 | 12 | 24 | 28 |
| 125 | | | | | | | | | | | | | | | |
| S. epidermidis | 26 | 32 | 34 | r | 18 | 24 | r | 18 | 24 | r | 8 | 14 | r | 14 | 18 |
| 563 | | | | | | | | | | | | | | | |
| S. epidermidis | 20 | 28 | 30 | r | 16 | 20 | r | 16 | 20 | r | 14 | 18 | r | 12 | 18 |
| 847 | | | | | | | | | | | | | | | |

¹Diameter of zone of inhibition in mm;
²r = resistant (no detectable zone of inhibition)

TABLE 8
Effectiveness of in vitro active ingredients in the “mouse infected
with Staphylococcus aureus” infection model

| | | Survival/ | Survival/ |
|---|---|---|---|
| Active ingredient | Tested | tested | controls |
| according to the | concentrations | mice n/n | n/n |
| invention | Staphylococcus aureus ATCC 6538 | | |
| 2a | 2 × 0.5 mg | 10/10 | 0/10 |
| | (25 mg/kg) | | |
| 1e | 2 × 0.5 mg | 3/3 | 0/5 |
| | (25 mg/kg) | | |
| 1f | 2 × 0.5 mg | 3/3 | 0/5 |
| | (25 mg/kg) | | |

### Example 20

### Targeted Increase in Lipophilicity of the Commercially Available Antibiotic Cefalexin (Formula 5, R1=H, R2=H, R3=CH₃, X═S) Through the Radical-Mediated Introduction of Novel Substitutes

To achieve sufficient fixing on a lipophilic surface, the lipophilicity of the commercially available antibiotic Cefalexin (Formula 5, R1=H, R2=H, R3=CH₃, X═S) can be increased selectively through the radical-mediated introduction of paradihydroxylated substitutes. Initially, the distribution behavior of Cefadroxil was set between an aqueous phase and a lipophilic phase.

Method cf. Example 12

### Result:

The determined logP value of the commercially available antibiotic Cefalexin (Formula 5, R1=H, R2=H, R3=CH₃, X═S) equals −2.63. In order to reach sufficient binding to a lipophilic bearing matrix, the lipophilicity should be increased selectively.

The problem was solved according to the invention through the radical-mediated reaction corresponding to Example 13 with 2,5-dihydroxy-N-(2-hydroxyethyl)benzamide (logP value=−1.21; Formula 6, R4=CONHCH₂CH₂OH).

An antibiotic with significantly higher lipophilicity (FIG. 9) was obtained while maintaining the antimicrobial activity (Table 7)

TABLE 9
logP value

| Active ingredient | |
|---|---|
| according to the | |
| invention | logP value |
| 1e | 1.61 |

### Example 21

### 3-chlorine-7-{2-[2-(2-hydroxyethylcarbamoyl)-3,6-dioxocyclohexa-1,4-dienylamino]-2-phenyl-acetylamino}-8-oxo-1-thia-5-azabicyclo[4.2.0]oct-3-en-4-carboxylic acid 1i

The active ingredient was obtained through the reaction of active ingredients of the general Formula 5 (R1=H, R2=H, R3=Cl, X═S) with substances of the general Formula 6 (R4=CONHCH₂CH₂OH).

### Structural Analysis

NMR spectra were recorded at 300 MHz ('H) and 75 MHz (13C and DEPT-135) in acetonitrile-d₃. ¹H NMR δ 3.36 (m, J=5.7 Hz, 2H, H-9″), 3.38 (d, J=18.2 Hz, 1H, H-2), 3.57 (t, J=5.6 Hz, 2H, H-10″), 3.75 (d, J=18.2 Hz, 1H, H-2), 4.99 (d, J=4.9 Hz, 1H, H-6), 5.72 (dd, J=4.8 Hz, J=9.0 Hz, 1H, H-7), 5.94 (d, J=6.7 Hz, 1H, H-13), 6.56 (d, J=10.1 Hz, 1H, H-4″), 6.71 (d, J=10.1 Hz, 1H, H-5″), 7.42 (m, 5H, H-2′, H-3′, H-4′, H-5′, H-6′), 7.66 (d, J=8.9 Hz, 1H, H-11), 9.76 (t, 1H, H-8″), 13.25 (d, 1H, J=6.2 Hz, H-14). ¹³C NMR δ 31.27 (C-2), 42.11 (C-9″), 58.09 (C-6), 60.04 (C-7), 61.48 (C-10″), 63.48 (C-13), 101.40 (C-1″), 124.60 (C-4), 128.41 (C-2′, C-6′), 129.13 (C-3), 129.84 (C-4′), 130.09 (C-3′, C-5′), 133.36 (C-4″), 138.69 (C-1′), 141.89 (C-5″), 153.55 (C-2″), 162.30 (C-8), 165.83 (C-9), 170.10 (C7″), 171.37 (C-12), 184.75 (C-6″), 185.51 (C-3″). LC/MS m/z 562.2 ([M+H]⁺, 584.1 [M+Na]⁺ API-ES pos. mode).

### Proof of Stability

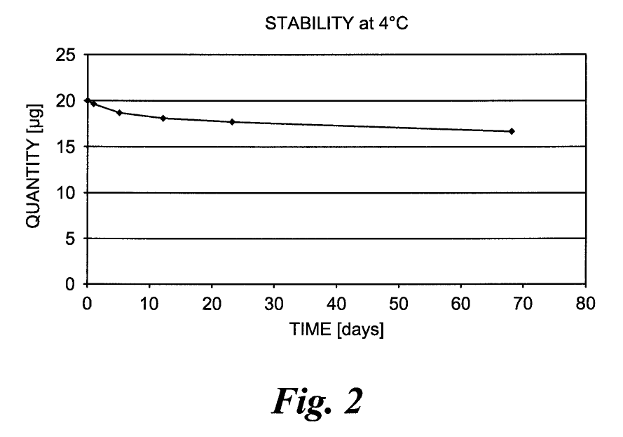

The novel active ingredient has proven to be stable in storage over a time period >60 days. FIG. 2 shows the storage stability of the 3-chlorine-7-{2-[2-(2-hydroxyethylcarbamoyl)-3,6-dioxocyclohexa-1,4-dienylamino]-2-phenyl-acetylamino}-8-oxo-1-thia-5-azabicyclo[4.2.0]oct-3-en-4-carboxylic acid 1i.

### Example 22

### 3-chlorine-7-[2-(2-carbamoyl-3,6-dioxocyclohexa-1,4-dienylamino)-2-phenyl-acetylamino]-8-oxo-1-thia-5-azabicyclo[4.2.0]oct-3-en-4-carboxylic acid 1j

The active ingredient was obtained through the reaction of active ingredients of the general Formula 5 (R1=H, R2=H, R3=Cl, X═S) with substances of the general Formula 6 (R4=CONH₂).

### Structural Analysis

NMR spectra were recorded at 300 MHz (1H) in acetonitrile-d₃.

¹H NMR δ 3.41 (d, J=18.4 Hz, 1H, H-2), 3.77 (d, J=18.4 Hz, 1H, H-2), 5.01 (d, J=4.9 Hz, 1H, H-6), 5.74 (dd, J=4.9 Hz, J=8.5 Hz, 1H, H-7), 5.96 (d, J=6.7 Hz, 1H, H-13), 6.60 (d, J=10.3 Hz, 1H, H-4″), 6.71 (d, J=10.3 Hz, 1H, H-5″), 7.45 (m, 5H, H-2′, H-3′, H-4′, H-5′, H-6′), 7.61 (d, J=8.5 Hz, 1H, H-11), 9.09 (s, 2H, H-8″), 13.27 (d, 1H, J=6.7 Hz, H-14). LC/MS m/z 518.2 ([M+H]⁺, 540.0 [M+Na]⁺ API-ES pos. mode).

### Example 23

### 3-chlorine-7-[2-(2-methoxycarbonyl-3,6-dioxocyclohexa-1,4-dienylamino)-2-phenyl-acetylamino]-8-oxo-1-thia-5-azabicyclo[4.2.0]oct-3-en-4-carboxylic acid 1k

The active ingredient was obtained through the reaction of active ingredients of the general Formula 5 (R1=H, R2=H, R3=Cl, X═S) with substances of the general Formula 6 (R4=COOCH₃).

### Structural Analysis

NMR spectra were recorded at 300 MHz (¹H) in acetonitrile-d₃. ¹H NMR δ 3.23 (d, J=18.4 Hz, 1H, H-2), 3.38 (d, J=18.4 Hz, 1H, H-2), 3.83 (br s, 3H, H-8″), 4.92 (d, J=4.6 Hz, 1H, H-6), 5.65 (dd, J=4.6 Hz, J=7.5 Hz, 1H, H-7), 5.96 (d, J=6.7 Hz, 1H, H-13), 6.68 (d, J=10.2 Hz, 1H, H-4″), 6.74 (d, J=10.1 Hz, 1H, H-5″), 7.42 (m, 5H, H-2′, H-3′, H-4′, H-5′, H-6′), 7.61 (d, J=7.5 Hz, 1H, H-11), 13.27 (d, 1H, J=6.7 Hz, H-14). LC/MS m/z 532.5 ([M+H]⁺, 554.5 [M+Na]⁺ API-ES pos. mode).

### Example 24

### 3-chlorine-7-[2-(2[ethoxycarbonyl-3,6-dioxocyclohexa-1,4-dienylamino)-2-phenyl-acetylamino]-8-oxo-1-thia-5-azabicyclo[4.2.0]oct-3-en-4-carboxylic acid 1l

The active ingredient was obtained through the reaction of active ingredients of the general Formula 5 (R1=H, R2=H, R3=Cl, X═S) with substances of the general Formula 6 (R4=COOCH₂CH₃).

### Structural Analysis

NMR spectra were recorded at 300 MHz (1H) in acetonitrile-d₃. ¹H NMR δ 1.14 (br, 3H, H-9″), 3.35 (d, J=18.2 Hz, 1H, H-2), 3.73 (d, J=18.2 Hz, 1H, H-2), 4.16 (br, 2H, H-8″), 4.95 (d, J=4.6 Hz, 1H, H-6), 5.69 (dd, J=4.6 Hz, J=8.8 Hz, 1H, H-7), 6.59 (d, J=10.2 Hz, 1H, H-4″), 6.70 (d, J=10.2 Hz, 1H, H-5″), 7.38 (m, 5H, H-2′, H-3′, H-4′, H-5′, H-6′), 7.55 (d, J=8.7 Hz, 1H, H-11). LC/MS m/z 547.0 ([M+H]⁺, 569.0 [M+Na]⁺ API-ES pos. mode).

### Example 25

### Antimicrobial Effect of the Novel Compounds According to Formula 4 (Example 21-Example 24) In Vitro

Methods cf. Example 5

### Results:

The novel active ingredients have a strong antimicrobial effect (Table 10).

### Example 26

### Production of Novel Cephalosporins Through the Formation of Salts with Cations that Themselves have an Active Ingredient Character

The fractions according to Example 6 were reacted with 7-aminocephalosporanic acid and also with active ingredients according to Formula 4 (R1=H, R2=H, R3=Cl, R4=CONHCH₂CH₂OH, X═S) and with active ingredients according to Formula 5 (R1=H, R2=H, R3=Cl, X═S). In this way, products with a strong antimicrobial effect were obtained.

### Example 27

### Antimicrobial Effectiveness In Vivo of the Novel Substances According to Formula 4 (Example 21-Example 24)

Methods cf. Example 11

### Result

In the “mouse infected with Staphylococcus aureus” infection model, the animals without effective treatment die at 100%. After treatment with the active ingredient according to the invention from Formula 4, all of the animals survive (Table 11).

TABLE 10
Antimicrobial effect of the novel active ingredients 1i to 1l and 2c.

| | Quantity n [mol] | | | | | | | | | | | | | | |
|---|---|---|---|---|---|---|---|---|---|---|---|---|---|---|---|
| | 2c | | | 1i | | | 1j | | | 1k | | | 1l | | |
| Strain | 0.019 | 0.1 | 0.19 | 0.029 | 0.14 | 0.29 | 0.02 | 0.1 | 0.2 | 0.029 | 0.14 | 0.29 | 0.019 | 0.09 | 0.19 |
| Bacillus | 36¹ | 38 | 40 | 26 | 32 | 34 | 28 | 36 | 40 | 30 | 36 | 38 | 30 | 34 | 38 |
| megaterium | | | | | | | | | | | | | | | |
| SBUG 1152 | | | | | | | | | | | | | | | |
| B. subtilis | 36 | 38 | 40 | 30 | 36 | 38 | 30 | 38 | 40 | 30 | 36 | 38 | 30 | 34 | 38 |
| AWD 166 | | | | | | | | | | | | | | | |
| Escherichia coli | 24 | 30 | 32 | 18 | 24 | 28 | 18 | 26 | 28 | 18 | 24 | 28 | 18 | 24 | 28 |
| SBUG 1135 | | | | | | | | | | | | | | | |
| Enterococcus | r² | 8 | 14 | r | r | r | r | 8 | 12 | r | 8 | 14 | r | r | r |
| faecalis 769 | | | | | | | | | | | | | | | |
| E. faecalis 945 | r | 8 | 12 | r | r | r | r | r | r | r | r | r | r | r | r |
| Staphylococcus | r | 10 | 12 | r | 10 | 14 | r | 8 | 12 | r | 12 | 14 | r | 10 | 14 |
| aureus 315 | | | | | | | | | | | | | | | |
| S. aureus 33490 | 24 | 30 | 32 | 14 | 22 | 26 | 16 | 24 | 26 | 16 | 24 | 26 | 14 | 20 | 24 |
| S. aureus 34289 | r | r | 10 | r | r | r | r | r | r | r | r | r | r | r | r |
| S. aureus 36881 | r | 12 | 18 | r | 12 | 18 | r | 12 | 16 | r | 14 | 18 | r | 8 | 18 |
| S. aureus 38418 | 26 | 34 | 38 | 20 | 30 | 34 | 22 | 30 | 34 | 22 | 30 | 34 | 18 | 28 | 30 |
| S. aureus 39105 | 22 | 24 | 28 | r | 18 | 22 | r | 16 | 18 | 8 | 20 | 22 | r | 18 | 22 |
| S. aureus 520 | r | 10 | 14 | r | r | r | r | 8 | 12 | r | r | 10 | r | r | 10 |
| S. aureus 526 | r | 8 | 12 | r | r | r | r | r | r | r | r | r | r | r | r |
| S. aureus | 30 | 38 | 40 | 26 | 34 | 36 | 28 | 34 | 38 | 28 | 36 | 38 | 24 | 30 | 34 |
| ATCC 6538 | | | | | | | | | | | | | | | |
| S. aureus North | r | 10 | 14 | r | 12 | 16 | r | 12 | 16 | r | 14 | 18 | r | 10 | 16 |
| German strain | | | | | | | | | | | | | | | |
| S. epidermidis | 22 | 24 | 30 | 12 | 18 | 22 | 16 | 22 | 26 | 14 | 22 | 24 | 14 | 20 | 22 |
| 1068 | | | | | | | | | | | | | | | |
| S. epidermidis | 28 | 30 | 32 | 14 | 22 | 26 | 18 | 26 | 28 | 18 | 24 | 28 | 16 | 24 | 28 |
| 1071 | | | | | | | | | | | | | | | |
| S. epidermidis | 26 | 34 | 36 | 20 | 28 | 32 | 22 | 30 | 32 | 20 | 30 | 32 | 18 | 26 | 28 |
| 125 | | | | | | | | | | | | | | | |
| S. epidermidis | 30 | 34 | 36 | 12 | 22 | 24 | 20 | 26 | 32 | 18 | 28 | 30 | 16 | 28 | 30 |
| 563 | | | | | | | | | | | | | | | |
| S. epidermidis | 14 | 24 | 28 | 12 | 20 | 24 | 10 | 20 | 24 | 8 | 24 | 28 | r | 20 | 22 |
| 847 | | | | | | | | | | | | | | | |

¹Diameter of zone of inhibition in mm;
²r = resistant (no detectable zone of inhibition)

TABLE 11
Effectiveness of in vitro active ingredients in the “mouse infected
with Staphylococcus aureus” infection model

| | | Survival/ | Survival/ |
|---|---|---|---|
| Active ingredient | Tested | tested | controls |
| according to the | concentrations | mice n/n | n/n |
| invention | Staphylococcus aureus ATCC 6538 | | |
| 2a | 2 × 0.5 mg | 10/10 | 0/10 |
| | (25 mg/kg) | | |
| 1i | 2 × 0.5 mg | 12/12 | 0/20 |
| | (25 mg/kg) | | |
| 1j | 2 × 0.5 mg | 3/3 | 0/5 |
| | (25 mg/kg) | | |
| 1k | 2 × 1.5 mg | 3/3 | 0/5 |
| | (75 mg/kg) | | |
| 1l | 2 × 1.0 mg | 3/3 | 0/5 |
| | (50 mg/kg) | | |

### Example 28

### Targeted Increase in the Lipophilicity of the Commercially Available Antibiotic Cefaclor (Formula 5, R1=H, R2=H, R3=Cl, X═S) Through the Radical-Mediated Introduction of Novel Substitutes

In order to reach sufficient fixing on a lipophilic surface, the lipophilicity of the commercially available antibiotic Cefaclor (Formula 5, R1=H, R2=H, R3=Cl, X═S) should be increased selectively through the radical-mediated introduction of paradihydroxylated substitutes. Initially, the distribution behavior of Cefaclor was set between an aqueous phase and a lipophilic phase.

Method cf. Example 12

### Result:

The determined logP value of the commercially available antibiotic Cefaclor (Formula 5, R1=H, R2=H, R3=Cl, X═S) equals −3.28. In order to achieve sufficient binding to a lipophilic bearing matrix, the lipophilicity should be increased selectively.

The problem was solved according to the invention through the radical-mediated reaction corresponding to Example 21 with 2,5-dihydroxy-N-(2-hydroxyethyl)benzamide (logP value=−1.21; Formula 5, R4=CONHCH₂CH₂OH).

An antibiotic was obtained with significantly higher lipophilicity (Table 12) while maintaining the antimicrobial activity (Table 10).

TABLE 12
logP value

| Active ingredient | |
|---|---|
| according to the | |
| invention | logP value |
| 1i | 1.81 |

### Example 29

### 3-chlorine-7-{2-[2-(2-hydroxyethylcarbamoyl)-3,6-dioxocyclohexa-1,4-dienylamino]-2-phenyl-acetylamino}-8-oxo-5-azabicyclo[4.2.0]oct-3-ene-4-carboxylic acid 1m

The active ingredient was obtained through the reaction of active ingredients of the general Formula 5 (R1=H, R2=H, R3=Cl, X═CH₂) with substances of the general Formula 6 (R4=CONHCH₂CH₂OH).

### Structural Analysis

NMR spectra were recorded at 300 MHz ('H) and 75 MHz (13C and DEPT-135) in acetonitrile-d₃. ¹H NMR δ 1.33 (m, 2H, H-1), 2.40 (m, 2H, H-2), 3.37 (m, J=5.6 Hz, 2H, H-9″), 3.58 (t, J=5.5 Hz, 2H, H-10″), 3.74 (m, J=5.0 Hz, 1H, H-6), 5.31 (m, J=5.0 Hz, J=8.3 Hz, 1H, H-7), 5.90 (d, J=6.7 Hz, 1H, H-13), 6.55 (d, J=10.2 Hz, 1H, H-4″), 6.66 (d, J=10.2 Hz, 1H, H-5″), 7.41 (m, 5H, H-2′, H-3′, H-4′, H-5′, H-6′), 7.54 (d, J=7.9 Hz, 1H, H-11), 9.76 (t, 1H, H-8″), 13.23 (d, 1H, J=6.3 Hz, H-14). ¹³C NMR δ 22.19 (C-1), 31.70 (C-2), 42.12 (C-9″), 53.13 (C-6), 59.24 (C-7), 61.51 (C-10″), 63.62 (C-13), 101.65 (C-1″), 124.74 (C-4), 128.33 (C-2′, C-6′), 129.03 (C-3), 129.79 (C-4′), 130.07 (C-3′, C-5′), 133.34 (C-4′), 138.96 (C-1′), 141.92 (C-5″), 153.47 (C-2″), 162.22 (C-8), 165.73 (C-9), 170.09 (C7″), 171.37 (C-12), 184.69 (C-6″), 185.49 (C-3″). LC/MS m/z 544.2 ([M+H]⁺, 566.1 [M+Na]⁺ API-ES pos. mode).

### Proof of Stability

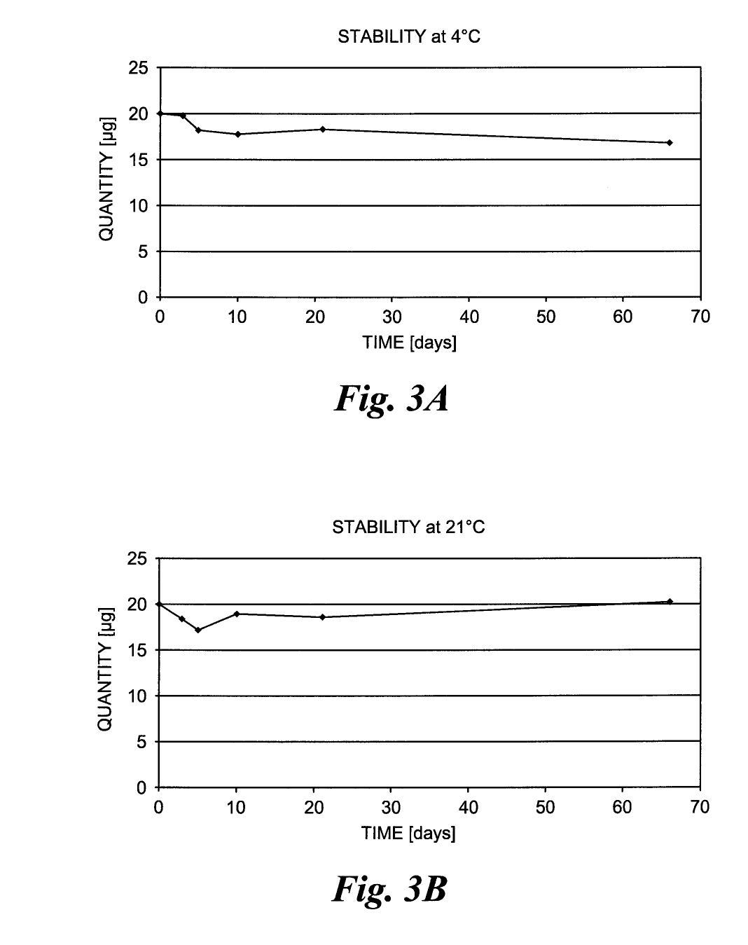

The novel active ingredient has proven to be stable in storage over a time period >60 days. FIG. 3 shows the storage stability of the 3-chlorine-7-{2-[2-(2-hydroxyethylcarbamoyl)-3,6-dioxocyclohexa-1,4-dienylamino]-2-phenyl-acetylamino}-8-oxo-5-azabicyclo[4.2.0]oct-3-ene-4-carboxylic acid 1m.

### Example 30

### 3-chlorine-7-[2-(2-carbamoyl-3,6-dioxocyclohexa-1,4-dienylamino)-2-phenyl-acetylamino]-8-oxo-5-aza-bicyclo[4.2.0]oct-3-ene-4-carboxylic acid 1n

The active ingredient was obtained through the reaction of active ingredients of the general Formula 5 (R1=H, R2=H, R3=Cl, X═CH₂) with substances of the general Formula 6 (R4=CONH₂).

### Structural Analysis

NMR spectra were recorded at 300 MHz (1H) in acetonitrile-d₃.

¹H NMR δ 1.34 (m, 2H, H-1), 2.40 (m, 2H, H-2), 3.72 (m, J=4.8 Hz, 1H, H-6), 5.35 (m, J=4.8 Hz, J=8.3 Hz, 1H, H-7), 5.90 (d, J=6.7 Hz, 1H, H-13), 6.55 (d, J=10.2 Hz, 1H, H-4″), 6.66 (d, J=10.2 Hz, 1H, H-5″), 7.41 (m, 5H, H-2′, H-3′, H-4′, H-5′, H-6′), 7.54 (d, J=8.4 Hz, 1H, H-11), 9.07 (s, 2H, H-8″), 13.14 (d, 1H, J=6.7 Hz, H-14). LC/MS m/z 500.3 ([M+H]⁺, 521.1 [M+Na]⁺ API-ES pos. mode).

### Example 31

### 3-chlorine-7-[2-(2-methoxycarbonyl-3,6-dioxocyclohexa-1,4-dienylamino)-2-phenyl-acetylamino]-8-oxo-5-aza-bicyclo[4.2.0]oct-3-ene-4-carboxylic acid 1o

The active ingredient was obtained through the reaction of active ingredients of the general Formula 5 (R1=H, R2=H, R3=Cl, X═CH₂) with substances of the general Formula 6 (R4=COOCH₃).

### Structural Analysis

NMR spectra were recorded at 300 MHz ('H) in acetonitrile-d₃. ¹H NMR δ 1.34 (m, 2H, H-1), 2.42 (m, 2H, H-2), 3.63 (br s, 3H, H-8″), 3.71 (m, J=4.9 Hz, 1H, H-6), 5.32 (m, J=4.9 Hz, J=8.3 Hz, 1H, H-7), 6.57 (d, J=10.1 Hz, 1H, H-4″), 6.67 (d, J=10.0 Hz, 1H, H-5″), 7.34 (m, 5H, H-2′, H-3′, H-4′, H-5′, H-6′), 7.91 (br d, 1H, H-11). LC/MS m/z 514.7 ([M+H]⁺, 535.9 [M+Na]⁺ API-ES pos. mode).

### Example 32

### 3-chlorine-7-[2-(2-ethoxycarbonyl-3,6-dioxocyclohexa-1,4-dienylamino)-2-phenyl-acetylamino]-8-oxo-5-azabicyclo[4.2.0]oct-3-ene-4-carboxylic acid 1p

The active ingredient was obtained through the reaction of active ingredients of the general Formula 5 (R1=H, R2=H, R3=Cl, X═CH₂) with substances of the general Formula 6 (R4=COOCH₂CH₃).

### Structural Analysis

NMR spectra were recorded at 300 MHz (¹H) in acetonitrile-d₃. ¹H NMR δ 1.13 (br, 3H, H-9″), 1.33 (m, 2H, H-1), 2.46 (m, 2H, H-2), 3.75 (m, J=5.1 Hz, 1H, H-6), 4.02 (br, 2H, H-8″), 5.34 (m, J=5.1 Hz, J=8.4 Hz, 1H, H-7), 6.59 (d, J=10.2 Hz, 1H, H-4″), 6.70 (d, J=10.0 Hz, 1H, H-5″), 7.38 (m, 5H, H-2′, H-3′, H-4′, H-5′, H-6′), 7.61 (br d, 1H, H-11). LC/MS m/z 529.8 ([M+H]⁺, 550.2 [M+Na]⁺ API-ES pos. mode).

### Example 33

### Antimicrobial Effect of the Novel Compounds According to Formula 4 (Example 29-Example 32) In Vitro

Methods cf. Example 5

### Result:

The novel active ingredients have a strong antimicrobial effect (Table 13).

TABLE 13
Antimicrobial effect of the novel active ingredients 1m to 1p and 2d.

| | Quantity n [mol] | | | | | | | | | | | | | | |
|---|---|---|---|---|---|---|---|---|---|---|---|---|---|---|---|
| | 2d | | | 1m | | | 1n | | | 1o | | | 1p | | |
| Strain | 0.019 | 0.094 | 0.19 | 0.018 | 0.092 | 0.18 | 0.019 | 0.096 | 0.19 | 0.019 | 0.094 | 0.19 | 0.019 | 0.094 | 0.19 |
| Enterococcus | r² | r | 8¹ | r | r | r | r | r | r | r | r | r | r | r | r |
| faecalis 769 | | | | | | | | | | | | | | | |
| Staphylococcus | r | r | 10 | r | r | 8 | r | r | 12 | r | r | 10 | r | r | 10 |
| aureus 315 | | | | | | | | | | | | | | | |
| S. aureus 36881 | r | 20 | 22 | r | 16 | 20 | r | r | 8 | r | r | 12 | r | 12 | 20 |
| S. aureus 38418 | 18 | 30 | >30 | 20 | 30 | >30 | 14 | 26 | 30 | 14 | 14 | 28 | 18 | 28 | >30 |
| S. aureus 520 | r | 10 | 14 | r | r | 8 | r | r | 8 | r | r | r | r | r | 8 |
| S. aureus | 28 | 34 | 38 | 22 | >30 | >30 | 22 | 28 | 32 | 22 | 28 | 30 | 22 | 30 | 32 |
| ATCC 6538 | | | | | | | | | | | | | | | |
| S. aureus North | r | r | 8 | r | r | 12 | r | r | 14 | r | r | 12 | r | r | 12 |
| German strain | | | | | | | | | | | | | | | |
| S. epidermidis | 18 | 24 | 32 | 20 | 26 | 28 | 14 | 22 | 26 | 14 | 20 | 24 | 16 | 26 | 28 |
| 125 | | | | | | | | | | | | | | | |
| S. epidermidis | 14 | 22 | 24 | r | 14 | 18 | r | 16 | 20 | r | 14 | 18 | r | 16 | 20 |
| 847 | | | | | | | | | | | | | | | |

¹Diameter of zone of inhibition in mm,
²r = resistant (no detectable zone of inhibition)

### Example 34

### Production of Novel Cephalosporins Through the Formation of Salts with Cations that Themselves have an Active Ingredient Character

The fractions according to Example 6 were reacted with 7-aminocephalosporanic acid and also with active ingredients according to Formula 4 (R1=H, R2=H, R3=Cl, R4=CONHCH₂CH₂OH, X═CH₂) and with active ingredients according to Formula 5 (R1=H, R2=H, R3=Cl, X═CH₂). In this way, products with a strong antimicrobial effect were obtained.

### Example 35

### Antimicrobial Effectiveness In Vivo of the Novel Substances According to Formula 4 (Example 29-Example 32)

Methods cf. Example 11

### Result

In the “mouse infected with Staphylococcus aureus” infection model, the animals without effective treatment die at 100%. After treatment with the active ingredient according to the invention from Formula 4, all of the animals survive (Table 14).

TABLE 14
Effectiveness of in vitro active ingredients in the “mouse infected
with Staphylococcus aureus” infection model

| | | Survival/ | Survival/ |
|---|---|---|---|
| Active ingredient | Tested | tested | controls |
| according to the | concentrations | mice n/n | n/n |
| invention | Staphylococcus aureus ATCC 6538 | | |
| 2a | 2 × 0.5 mg | 10/10 | 0/10 |
| | (25 mg/kg) | | |
| 1m | 2 × 0.5 mg | 9/9 | 0/15 |
| | (25 mg/kg) | | |
| 1n | 2 × 1.0 mg | 3/3 | 0/5 |
| | (50 mg/kg) | | |
| 1o | 2 × 1.0 mg | 2/3 | 0/5 |
| | (50 mg/kg) | | |
| 1p | 2 × 1.0 mg | 2/3 | 0/5 |
| | (50 mg/kg) | | |

### Example 36

### Targeted Increase in Lipophilicity of the Commercially Available Antibiotic Loracarbef (Formula 5, R1=H, R2=H, R3=Cl, X═CH₂) Through the Radical-Mediated Introduction of Novel Substitutes

In order to achieve sufficient fixing on a lipophilic surface, the lipophilicity of the commercially available antibiotic Loracarbef (Formula 5, R1=H, R2=H, R3=Cl, X═CH₂) should be increased selectively through the radical-mediated introduction of paradihydroxylated substitutes. Initially, the distribution behavior of Loracarbef was set between an aqueous phase and a lipophilic phase.

Method cf. Example 12

### Result:

The determined logP value of the commercially available antibiotic Loracarbef (Formula 5, R1=H, R2=H, R3=Cl, X═CH₂) equals −3.13. In order to achieve sufficient binding to a lipophilic bearing matrix, the lipophilicity should be increased selectively.

The problem was solved according to the invention through the radical-mediated reaction corresponding to Example 29 with 2,5-dihydroxy-N-(2-hydroxyethyl)benzamide (logP value=−1.21; Formula 6, R4=CONHCH₂CH₂OH).

An antibiotic was obtained with significantly higher lipophilicity (Table 12) while maintaining the antimicrobial activity (Table 15).

TABLE 15
logP value

| Active ingredient | |
|---|---|
| according to the | |
| invention | logP value |
| 1m | 1.62 |

The structural analysis and the tests for the biological activity of Example 37-Example 41 form the basis of the designations of the structural elements and also the substances corresponding to Formula 11.

### Example 37

### 7-[2-(5-methyl-3,4-dioxocyclohexa-1,5-dienylamino)-2-(4-hydroxyphenyl)-acetylamino]-cephalosporanic acid 1q

The active ingredient was obtained through the reaction of active ingredients of the general Formula 5 (R1=OH, R2=H, R3=CH₃, X═S) with substances of the general Formula 9 (R4=H, R5=CH₃).

### Structural Analysis

NMR spectra were recorded at 300 MHz ¹H and HMBC and HSQC in DMSO-d₆.

¹H NMR δ (DMSO-d₆) 1.86 (s, 3H, H7″), 1.99 (s, 3H, H10), 3.29 (d, J=18.4 Hz, 1H, H2), 3.49 (d, J=18.4 Hz, 1H, H2), 4.89 (d, J=4.4 Hz, 1H, H6), 5.19 (d(s), J=1.5 Hz, 1H, H2″), 5.31 (d, J=7.0 Hz, 1H, H13), 5.65 (dd, J=4.7 Hz, J=8.1 Hz, 1H, H7), 6.76 (d, J=8.2 Hz, 2H, H3′, H5′), 7.14 (d(s), J=1.5 Hz, 1H, H6″), 7.27 (d, J=8.2 Hz, 2H, H2′, H6′), 8.71 (d, J=7.1 Hz, 1H, H14), 9.29 (d, J=8.3 Hz, 1H, H11). ¹³C NMR δ (DMSO-d₆) 15.6 (C7″), 19.7 (C10), 29.1 (C2), 57.1 (C6), 58.8 (C7), 59.5 (C13), 95.1 (C2″), 115.7 (C3′, C5′), 122.9 (C4), 126.8 (C1′), 128.9 (C2′, C6′), 129.6 (C3), 133.7 (C6″), 140.0 (C5″), 155.2 (C1″), 157.8 (C4′), 163.8 (C9), 164.1 (C8), 170.0 (C12), 174.7 (C3″), 183.6 (C4″). LC/MS m/z 483.4 ([M]⁺, 504.8 [M+Na]⁺ API-ES pos. mode)

### Example 38

### 7-[2-(6-methyl-3,4-dioxocyclohexa-1,5-dienylamino)-2-(4-hydroxyphenyl)-acetylamino]-cephalosporanic acid 1r

The active ingredient was obtained through the reaction of active ingredients of the general Formula 5 (R1=OH, R2=H, R3=CH₃, X═S) with substances of the general Formula 9 (R4=CH₃, R5=H).

### Structural Analysis

NMR spectra were recorded at 300 MHz ¹Hand HMBC and HSQC in DMSO-d₆.

¹H NMR δ (DMSO-d₆) 1.99 (s, 3H, H10), 2.32 (s, 3H, H8″), 3.29 (d, J=18.1 Hz, 1H, H2), 3.48 (d, J=18.4 Hz, 1H, H2), 4.99 (d, J=4.4 Hz, 1H, H6), 5.12 (s, 1H, H2″), 5.25 (d, J=6.1 Hz, 1H, H13), 5.69 (dd, J=4.9 Hz, J=7.8 Hz, 1H, H7), 6.32 (s, 1H, H5″), 6.76 (d, J=8.2 Hz, 2H, H3′, H5′), 7.11 (d, J=6.1 Hz, 1H, H14), 7.34 (d, J=8.2 Hz, 2H, H2′, H6′), 9.29 (d, J=8.4 Hz, 1H, H11). ¹³C NMR δ (DMSO-d₆) 17.5 (C7″), 19.5 (C10), 28.6 (C2), 56.8 (C6), 58.8 (C7), 59.5 (C13), 97.7 (C2″), 115.9 (C3′, C5′), 123.0 (C4), 126.7 (C1′), 128.7 (C2′, C6′), 129.4 (C5″), 129.6 (C3), 147.2 (C6″), 154.3 (C1″), 158.0 (C4′), 163.6 (C9), 163.9 (C8), 170.2 (C12), 175.6 (C3″), 182.8 (C4″). LC/MS m/z 483.4 ([M]⁺, 504.9 [M+Na]⁺ API-ES pos. mode).

### Example 39

### 3-chlorine-7-[2-(5-methyl-3,4-dioxocyclohexa-1,5-dienylamino)-2-phenyl-acetylamino]-8-oxo-5-azabicyclo[4.2.0]oct-3-ene-4-carboxylic acid 1s

The active ingredient was obtained through the reaction of active ingredients of the general Formula 5 (R1=H, R2=H, R3=Cl, X═CH₂) with substances of the general Formula 9 (R4=CH₃, R5=H).

### Structural Analysis

NMR spectra were recorded at 300 MHz ¹H and HMBC and HSQC in DMSO-d₆.

¹H NMR δ (DMSO-d₆) 1.35 (m, 2H, H1), 1.86 (s, 3H, H7″), 2.48 (m, 2H, H2), 3.72 (m, J=7.8 Hz, 1H, H6), 5.16 (s, 1H, H2″), 5.25 (dd, J=7.9 Hz, J=5.6 Hz, 1H, H7), 5.33 (s, 1H, H13), 7.19 (s, 1H, H6″), 7.35 (dd, J=7.4 Hz, 1H, H4′), 7.40 (dd, J=7.4 Hz, 2H, H3′, H5′), 7.49 (d, J=7.6 Hz, 2H, H2′, H6′), 8.88 (broad, 1H, H14), 9.39 (d, J=8.0 Hz, 1H, H11). ¹³C NMR δ (DMSO-d₆) 21.5 (C1), 28.0 (C2), 51.4 (C6), 57.7 (C7), 60.1 (C13), 95.0 (C2″), 120.2 (C4), 127.2 (C4′), 127.6 (C2′, C6′), 128.5 (C3), 128.7 (C3′, C5′), 133.5 (C6″), 136.5 (C1′), 163.9 (C8), 169.1 (C12), 183.0 (C4″). LC/MS m/z 469.5 ([M]⁺, 491.3 [M+Na]⁺ API-ES pos. mode).

### Example 40

### Antimicrobial Effect of the Novel Compounds According to Formula 8 (Example 37-Example 39) In Vitro

Methods cf. Example 5

### Results:

The novel active ingredients have a strong antimicrobial effect (Table 16).

TABLE 16
Antimicrobial effect of the novel active ingredients 1q to 1r, 2a, 2b, and 4a, 4b.

| Active | Quantity | Enterococcus | Staphylococcus | S. aureas | S. aureas | S. aureus |
|---|---|---|---|---|---|---|
| ingredient | n [mol] | faecalis 769 | aureus 315 | 36881 | 38418 | 520 |
| 1q | 0.02 | r¹ | r | r | 10² | r |
| | 0.1 | r | 10 | r | 18 | r |
| | 0.2 | 10 | 14 | 10 | 22 | 10 |
| 1r | 0.02 | r | r | r | 12 | r |
| | 0.1 | r | r | r | 22 | r |
| | 0.2 | r | 10 | 12 | 26 | r |
| 1s | 0.02 | r | r | r | 14 | r |
| | 0.1 | r | 8 | r | 24 | r |
| | 0.2 | r | 12 | 10 | 28 | 10 |
| 2a | 0.02 | r | r | r | 20 | r |
| | 0.1 | 10 | r | 14 | 30 | r |
| | 0.2 | 16 | r | 26 | >30 | 12 |
| 2d | 0.02 | r | 16 | r | 24 | r |
| | 0.1 | r | 20 | 20 | 30 | 8 |
| | 0.2 | 12 | 22 | 28 | >30 | 14 |
| 4a | 0.02 | r | r | r | r | r |
| | 0.1 | r | r | r | r | r |
| | 0.2 | r | r | r | r | r |
| 4b | 0.02 | r | r | r | r | r |
| | 0.1 | r | r | r | r | r |
| | 0.2 | r | r | r | r | r |

| | | S. aureus | | | |
|---|---|---|---|---|---|
| Active | S. aureus | North German | S. epidermidis | S. epidermidis | S. epidermidis |
| ingredient | ATCC 6538 | strain | 125 | 535 | 847 |
| 1q | 16 | r | 12 | r | r |
| | 20 | 10 | 20 | r | 14 |
| | 26 | 12 | 26 | 10 | 16 |
| 1r | 18 | r | 16 | r | r |
| | 30 | r | 26 | r | 16 |
| | >30 | 12 | 28 | r | 18 |
| 1s | 20 | r | 14 | r | r |
| | 30 | 8 | 22 | r | 14 |
| | >30 | 14 | 26 | 10 | 18 |
| 2a | 30 | r | 22 | r | 10 |
| | >30 | r | 30 | r | 22 |
| | >30 | r | >30 | r | 26 |
| 2d | 28 | r | 22 | r | 10 |
| | >30 | r | 30 | r | 20 |
| | >30 | 8 | >30 | r | 24 |
| 4a | r | r | r | r | r |
| | r | r | r | r | r |
| | r | 10 | 8 | r | 10 |
| 4b | r | r | r | r | r |
| | r | r | r | r | r |
| | r | 10 | r | r | r |

¹r = resistant (no detectable zone of inhibition)
²Diameter of zone of inhibition in mm

### Example 41

### Antimicrobial Effectiveness In Vivo of the Novel Substances According to Formula 8 (Example 37)

Methods cf. Example 11

### Result

In the “mouse infected with Staphylococcus aureus” infection model, the animals without effective treatment die at 100%. After treatment with the active ingredient according to the invention from Formula 8, at least 50% of the animals survive (Table 17).

TABLE 17
Effectiveness of in vitro active ingredients in the “mouse infected
with Staphylococcus aureus” infection model

| | | Survival/ | Survival/ |
|---|---|---|---|
| Active ingredient | Tested | tested | controls |
| according to the | concentrations | mice n/n | n/n |
| invention | Staphylococcus aureus ATCC 6538 | | |
| 2a | 2 × 0.5 mg | 10/10 | 0/10 |
| | (25 mg/kg) | | |
| 1q | 2 × 0.5 mg | 3/6 | 0/10 |
| | (25 mg/kg) | | |
| | 2 × 1.0 mg | 1/3 | 0/5 |
| | (50 mg/kg) | | |

### Example 42

### Material of the Hydrophilic Absorbent Core of a Wound Dressing with Active Ingredients According to Structural Formula 3

A layer of the absorbent core was cut into 1 cm²-large pieces. These were saturated with suspensions of different concentrations of the active ingredient according to Structural Formula 3 and tested in the agar test for germ reduction. For this purpose, the germ suspension was plated linearly with the help of the Whitley Automatic Spiral Plater (WASP). In this way, agar plates with a uniform bacteria growth. After 30 min, the areas on which the wound dressing was to be applied were marked and the control surfaces were set precisely. 25 μl saline solution was dripped onto each of these areas. Uncoated compress material and wound dressings coated with the substances according to the invention were set and lightly pressed onto these drops. The plates were let stand for 15 min for pre-diffusion at room temperature until they were placed in the incubation cabinet (37° C.). After 24 h, the surfaces of the wound dressings were marked. The bacteria was counted on the surfaces underneath the wound dressings and in the control areas that had been given only saline solution. The wound dressings were transferred to fresh agar, in order to count possibly surviving germs.

Laying of the absorbent core on a non-inoculated agar plate. At these low germ counts, colonization was calculated only through statistical methods. Under the assumption of a Poisson distribution, the count to be expected in the middle for surviving germs m is calculated according to the formula

m=−ln p/pₒ

(p=number of tests without detection of germs, pₒ=total number of tests).

### Results

Uncoated wound dressings lead, as expected, to a strong germ propagation.

In contrast, for wound dressings coated with the active ingredient according to Structural Formula 3 at a concentration of 0.02%, the bacteria growth under the dressing was completely suppressed. However, only a zone of inhibition of 0.93±0.73 mm (n=7) around the wound dressing was observed, i.e., the diffusion into areas around the wound dressing was minimal. After transfer to fresh agar, in 3 cases, a small bacteria growth was observed and, in 7 cases, no bacteria growth was observed, i.e., on average, only 0.35 surviving germs were to be expected at this concentration of the novel polyhexanide active ingredient according to Structural Formula 3.

For a reduction of the concentration of the active ingredient to 0.01%, at the statistical average, 0.2 KBE was to be expected under the wound dressings (n=5). After bringing the absorbent core onto non-inoculated agar, in 2 cases, small growth and, in 3 cases, strong growth was observed, i.e., at this concentration, only bacteriostasis and no bactericidal effect was achieved.

For an increase of the concentration to 0.04%, as expected there occurred no growth under the wound dressing. For transfer to non-inoculated agar, at the statistical average, a germ count of 0.22 (n=5) was observed. However, the zone of inhibition around the absorbent core rose to 3 mm, i.e., at this concentration, diffusion into the surrounding medium began.

### Example 43

### Testing of the Wound Dressing Coated with Active Ingredients According to Structural Formula 2 or 3 in the Filter Test

Different size pieces of the different wound dressings were placed in a funnel and germ suspension (200 000 KbE/ml) was dripped at a rate of approximately 20 drops/min (Fig.). The first 2 ml of the filtrate were captured. After dilution, the germ count was determined in the filtrate with the spiral plater WASP and compared with the original germ count. The dilution was here adapted each time to the original germ count.

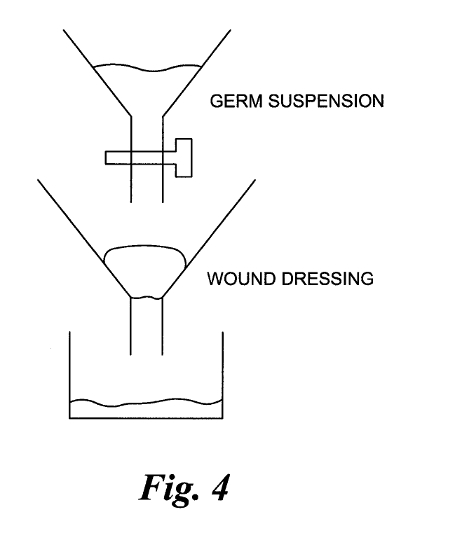

FIG. 4 shows the schematic diagram of the filter test setup.

### Result

For uncoated wound dressings, the germs has almost completely recovered. In contrast, for wound dressings coated with the active ingredient according to Structural Formula 2 or 3, all of the germs had been inactivated; no germ growth was detected in the captured filtrate.

### Example 44

The active ingredient according to Structural Formula 2 was mixed with a mixture of different fatty acids (tetradecane acid, pentadecane acid, hexadecane acid, cis-9-hexadecane acid, octadecane acid, cis-9-octadecane acid, cis-9,12-octadecane acid obtained through n-hexane extraction from the microalgae extractor DIONEX ASE 200). With the obtained product, absorbent compresses were coated with a concentration of 0.01%.

### Results

The germ growth could be stopped completely. Mixtures of natural fatty acids could be used according to claim 11 as acids for the transformation of the substance according to Structural Formula 2. The obtained product is distinguished by a strong antimicrobial effectiveness.

### Example 45

### Testing on Pig Skin

### Methodology

The commercially available active ingredient according to Structural Formula 1, amoxicillin, and the salt according to claim 2 with amoxicillin as the anion, each in a concentration of 1%, were worked into a W/O emulsion base.

The tests with these formulations were performed on the skin of a Vietnamese pot-bellied pig immediately after slaughter.

The skin was colonized with Staph. aureus (North German epidemic strain). Then the ointments with the corresponding active ingredients were applied to marked skin areas. After 24 h, the corresponding skin areas were rinsed and the germ count in the rinsed solution was determined.

### Results

While germs were still detectable in both of the individual substances, the product according to the invention led to a complete inactivation of the germs.

TABLE 18

| Ointment | Colony-forming units |
|---|---|
| Base W/O | >100 000 |
| Base W/O + active ingredient | 23 |
| according to Formula 1 | |
| Base W/O + amoxicillin | 52 |
| Base W/O + active ingredient | 0 |
| according to Claim 2 | |

### Example 46

### Working into a Cellulose Gel

### Methodology

The commercially available active ingredient according to Structural Formula 1, amoxicillin, and the salt according to claim 2 with amoxicillin as the anion, each in a concentration of 0.1 and 1%, were worked into an alcohol-free cellulose gel.

The testing was performed according to Example 45.

### Results

The formulations according to the invention are stable.

In contrast to the individual substances, with the formulation according to the invention, a complete inactivation of the multi-resistant Staph. aureus strain can be achieved.

TABLE 19

| Ointment | Colony-forming units | |
|---|---|---|
| Active ingredient content | 0.1% | 1% |
| Base cellulose-gel | 507 | 403 |
| Base + active ingredient | 77 | 15 |
| according to Formula 1 | | |
| Base + amoxicillin | 57 | 64 |
| Base W/O + active ingredient | 24 | 0 |
| according to Claim 2 | | |

### Example 47

### Tests of the Formulations According to Example 46 on Oral Mucosa

### Methodology

see Example 45, but oral and pharyngeal mucosa were used.

### Results

The formulation according to the invention also exhibited the best effectiveness on mucosa.

TABLE 20

| Ointment | Colony-forming units | |
|---|---|---|
| Active ingredient content | 0.1% | 1% |
| Base cellulose-gel | 149 | 170 |
| Base + active ingredient | 23 | 45 |
| according to Formula 1 | | |
| Base + amoxicillin | 117 | 28 |
| Base W/O + active ingredient | 2 | 0 |
| according to Claim 2 | | |

22. A β-lactam derivative of the general formula 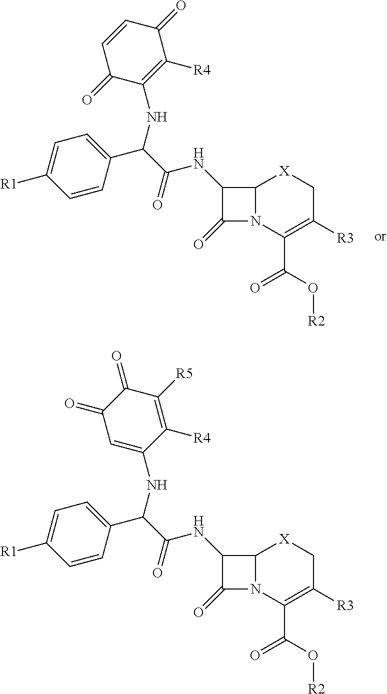
with:
R1=H, OH
R2=H, Na, CH₂OH, CHCH₃OCOOC₂H₅, CHCH₃OCOOCH(CH₃)₂, CH₂OCOC(CH₃)₃
R3=CH₃, Cl
R4=CONHCH₂CH₂OH, CONH₂, COOCH₃, COOCH₂CH₃, COOH, COCH₃, CHO, CH₃, CH₂(CH₂)₀₋₂₀CH₃, C(CH₃)₃, C₆H₅, Cl, Br, OCH₃, O(CH₂)₀₋₂₀CH₃
R5=CONHCH₂CH₂OH, CONH₂, COOCH₃, COOCH₂CH₃, COOH, COCH₃, CHO, CH₃, CH₂(CH₂)₀₋₂₀CH₃, C(CH₃)₃, C₆H₅, Cl, Br, OCH₃, O(CH₂)₀₋₂₀CH₃
X═S, CH₂.

23. A salt whose anionic component originates from a
a). β-lactam derivative according to claim 22 or
b) commercially available β-lactam derivative or
c) derivative of 6-aminopenicillanic acid or
d) derivative of 7-aminocephalosporanic acid
and whose cationic component is polyhexamethylene biguanide.

24. The salt according to claim 22, wherein it comprises substances of the general formula 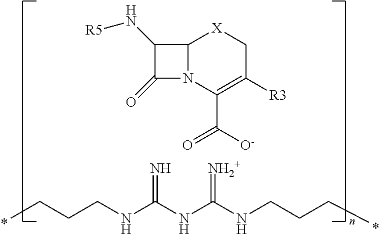
wherein
R3=CH₃, Cl; X═S, CH₂ 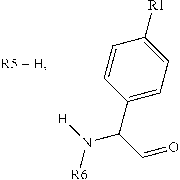
R1=H, OH; 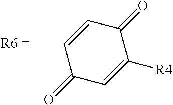
or H
R4=CONHCH₂CH₂OH, CONH₂, COOCH₃, COOCH₂CH₃, COOH, COCH₃, CHO, CH₃, CH₂(CH₂)₀₋₂₀CH₃, C(CH₃)₃, C₆H₅, Cl, Br, OCH₃, O(CH₂)₀₋₂₀CH₃.

25. The salt according to claim 23, wherein n=2-5.

26. The salt according to claim 24, wherein n=2-5.

27. A polyhexamethylene biguanide hydrogen carbonate as intermediate product in the production of the polyhexamethylene biguanide salt according to claim 23.

28. A polyhexamethylene biguanide hydrogen carbonate as intermediate product in the production of the polyhexamethylene biguanide salt according to claim 24.

29. A method for the production of β-lactam derivatives according to claim 22, wherein β-lactam antibiotics with a free amino group according to Formula 5 with substrates of polyphenol oxidases whose hydroxy groups have an ortho arrangement are substituted on the amino group under the influence of
a) enzymes of the classification EC 1.10.3.2 and/or
b) peroxidases of the classification EC 1.11.17 and/or
c) monophenol monooxygenases EC 114.99.1 and/or
d) ascorbate oxidases EC 1.10.3.3.

30. A method for the production of β-lactam derivatives according to claim 22, wherein cephalosporins with a free amino group according to Formula 5 with substrates of polyphenol oxidases are substituted on the amino group under the influence of
a) enzymes of the classification EC 1.10.3.2 and/or
b) peroxidases of the classification EC 1.11.17 and/or
c) monophenol monooxygenases EC 114.99.1 and/or
d) ascorbate oxidases EC 1.10.3.3.

31. A method for the production of salts according to claim 23, comprising the following steps:
reaction of a soluble salt of polyhexamethylene biguanide with an alkali or ammonium hydrogen carbonate, wherein polyhexamethylene biguanide hydrogen carbonate precipitates out; and
reaction of the polyhexamethylene biguanide hydrogen carbonate with a β-lactam derivative according to one of (a) to (d).

32. The method according to claim 31, wherein the precipitation of the polyhexamethylene biguanide hydrogen carbonate is performed in fractions to obtain polyhexamethylene biguanide hydrogen carbonate of different molecular weight ranges.

33. The method for the production of polyhexamethylene biguanide hydrogen carbonate as an intermediate product and its separation into molecular weight ranges, wherein polyhexamethylene biguanide hydrochloride is precipitated out in aqueous solution with alkali hydrogen carbonate, wherein the precipitation is realized partially and step by step to perform fractionation in narrow molecular weight ranges of the polymers.
